(19) United States
(12) Patent Application Publication
(10) Pub. No.: US 2005/0163848 A1
(43) Pub. Date: Jul. 28, 2005

(54) Compositions and dosage forms for enhanced absorption of gabapentin and pregabalin

(76) Inventors: Patrick S.L. Wong, Burlingame, CA (US); Dong Yan, Cupertino, CA (US); George V. Guittard, Cupertino, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/978,136

(22) Filed: Oct. 29, 2004

Related U.S. Application Data
(60) Provisional application No. 60/516,259, filed on Oct. 31, 2003.
(60) Provisional application No. 60/519,509, filed on Nov. 12, 2003.

(51) Int. Cl. A61K 31/195; A61K 31/185; A61K 9/24

(52) U.S. Cl. 424/473; 514/561; 514/553

(57) ABSTRACT

A complex comprised of gabapentin or pregabalin and a transport moiety, such as an alkyl sulfate, is described. The complex has an enhanced absorption in the gastrointestinal tract, particularly the lower gastrointestinal tract. The complex, and compositions and dosage forms prepared using the complex, provide for absorption by the body of the drug through a period of ten to twenty-four hours, thus enabling a once-daily dosage form for gabapentin or pregabalin.

### CROSS-REFERENCE TO RELATED APPLICATIONS

This application claims the benefit of U.S. provisional patent application No. 60/516,259, filed Oct. 31, 2003, and of U.S. provisional patent application No. 60/519,509, filed Nov. 12, 2003, both applications are incorporated herein by reference in their entirety.

### FIELD OF THE INVENTION

This invention relates to the compositions and dosage forms for delivery of gabapentin or pregabalin. More particularly, the invention relates to a complex of gabapentin or pregabalin and a transport moiety where the complex provides an enhanced absorption of the drug in the gastrointestinal tract, and more particularly, in the lower gastrointestinal tract.

### BACKGROUND OF THE INVENTION

Scientific understanding about the pathogenesis of neuropathic pain has grown over the last decades as basic research with animal models of neuropathic pain and human clinical trials have revealed the pathophysiological and biochemical changes in the nervous system due to an insult or disease (Backonja, M. M., Clin. J. Pain, 16(2):S67-72 (2000)). Neuropathic pain is a chronic pain, often experienced by cancer patients, stroke victims, elderly persons, diabetics, as painful diabetic neuropathy, persons with herpes zoster (shingles), as postherpetic neuralgia, and in persons with neurodegenerative diseases, such as amyotrophic lateral sclerosis (ALS). Clinical characteristics of neuropathic pain include burning, spontaneous pain, shooting pain, and evoked pains. Distinct pathophysiological mechanisms lead to specific sensory symptoms, such as dynamic mechanical allodynia and cold hyperalgesia.

Therapies for treatment of neuropathic pain include use of traditional pain agents such as nonsteroidal anti-inflammatory drugs, analgesics, opoids, or tricyclic antidepressants (Max, M. B., Ann. Neurol., 35(Suppl):S50-S53 (1994); Raja, S. N. et al., Neurology, 59:1015 (2002); Galer, B. S. et al., Pain, 80:533 (1999)). Many patients are refractory to these and other treatments because of inadequate pain relief or intolerable side effects.

The anticonvulsant gabapentin has a clearly demonstrated analgesic effect for the treatment of neuropathic pain, and specifically for the treatment of painful diabetic neuropathy and postherpetic neuralgia (Wheeler, G., Curr. Opin. Invest. Drugs, 3(3):470 (2002)). Gabapentin is also an effective medication for controlling some types of seizures, particularly seizures resulting from epilepsy (Johannessen, S. I. et al., Ther. Drug Monitoring, 25:347 (2003)). Similarly, pregabalin has been shows to be effective for treatment of postherpetic neuralgia and painful diabetic neuropathy (Dworkin, R. H. et al., Neurology, 60:1274 (2003)).

Gabapentin is absorbed from the proximal small bowel into the blood stream by the L-amino acid transport system (Johannessen, supra at 350). Bioavailability of the drug is dose dependent, apparently because the L-amino acid transport system saturates, limiting the amount of drug absorbed (Stewart, B. H. et al., Pharm. Res., 10:276 (1993)). For example, serum gabapentin concentrations increase linearly with doses up to about 1800 mg/d, and then continue to increase at higher doses but less than expected, possibly because the absorption mechanism from the upper G.I. tract becomes saturated (Stewart, supra.).

The L-amino transport system responsible for absorption of gabapentin is present primarily in the epithelial cells of the small intestine (Kanai, Y. et al., J. Toxicol. Sci., 28(1):1 (2003)), thus limiting the absorption of the drug. Pregabalin also appears to be absorbed by the L-amino transport system, along with other amino acid transport systems ((Dworkin, supra, p. 1282).

Differences in the cellular characteristics of the upper and lower G.I. tracts also contribute to the poor absorption of molecules in the lower G.I tract. FIG. 1 illustrates two common routes for transport of compounds across the epithelium of the G.I. tract. Individual epithelial cells, represented by 10a, 10b, 10c, form a cellular barrier along the small and large intestine. Individual cells are separted by water channels or tight junctions, such as junctions 12a, 12b. Transport across the epithelium occurs via either or both a transcellular pathway and a paracellular pathway. The transcellular pathway for transport, indicated in FIG. 1 by arrow 14, involves movement of the compound across the wall and body of the epithelial cell by passive diffusion or by carrier-mediated transport. The paracellular pathway of transport involves movement of molecules through the tight junctions between individual cells, as indicated by arrow 16. Paracellular transport is less specific but has a much greater overall capacity, in part because it takes place throughout the length of the G.I. tract. However, the tight junctions vary along the length of the G.I tract, with an increasing proximal to distal gradient in effective ‘tightness’ of the tight junction. Thus, the duodenum in the upper G.I. tract is more “leaky” than the ileum in the upper G.I. tract which is more “leaky” than the colon in the lower G.I. tract (Knauf, H. et al., Klin. Wochenschr., 60(19):1 191-1200 (1982)).

Since the typical residence time of a drug in the upper G.I. tract is from approximately four to six hours, drugs having poor colonic absorption are absorbed by the body through a period of only four to six hours after oral ingestion. Frequently it is medically desirable that the administered drug be presented in the patient's blood stream at a relatively constant concentration throughout the day. To achieve this with traditional drug formulations that exhibit minimal colonic absorption, patients would need to ingest the drugs three to four times a day. Practical experience with this inconvenience to patients suggests that this is not an optimum treatment protocol. Accordingly, it is desired that a once daily administration of such drugs, with long-term absorption throughout the day, be achieved.

To provide constant dosing treatments, conventional pharmaceutical development has suggested various controlled release drug systems. Such systems function by releasing their payload of drugs over an extended period of time following administration. However, these conventional forms of controlled release systems are not effective in the case of drugs exhibiting minimal colonic absorption. Since the drugs are only absorbed in the upper G.I. tract and since the residence time of the drug in the upper G.I. tract is only four to six hours, the fact that a proposed controlled release dosage form may release its payload after the residence period of the dosage form in the upper G.I. does not mean the that body will continue to absorb the controlled release drug past the four to six hours of upper G.I. residence. Instead, the drug released by the controlled release dosage form after the dosage form has entered the lower G.I. tract is generally not absorbed and, instead, is expelled from the body with other matter from the lower G.I.

The use of gabapentin to control seizures or neuropathic pain would be greatly improved if an effective concentration of the drug were present in the patient's blood stream throughout the day. To achieve this with traditional gabapentin formulations, patients would need to ingest gabapentin dosages three to four times a day. Practical experience with this inconvenience to patients suggests that this is not an optimum treatment protocol. Additionally, a true once-daily gabapentin treatment would provide advantages beyond convenience. Numerous other advantages are provided by a relatively constant dosage of gabapentin in the bloodstream of the patient. Accordingly, it is desired that a once daily administration of gabapentin, with long-term absorption throughout the day, be achieved.

### SUMMARY OF THE INVENTION

Accordingly, in one aspect the invention includes a substance comprised of gabapentin or pregabalin and a transport moiety, the gabapentin or pregabalin and the transport moiety forming a complex.

In one embodiment, the transport moiety is an alkyl sulfate salt having between 6-12 carbon atoms. A preferred alkyl sulfate salt is a lauryl sulfate salt.

In another aspect, the invention includes a composition, comprising, a complex comprised of gabapentin or pregabalin and a transport moiety, and a pharmaceutically acceptable vehicle, wherein the composition has an absorption in the lower gastrointestinal tract at least 5-fold higher than gabapentin or pregabalin.

In another aspect, the invention includes a one embodiment, dosage form comprising the composition described above or the substance described above.

In one embodiment, the dosage form is an osmotic dosage form. Exemplary dosage forms, in one embodiment, have (i) a push layer; (ii) drug layer comprising a gabapentin-transport moiety complex or a pregabalin-transport moiety complex; (iii) a semipermeable wall provided around the push layer and the drug layer; and (iv) an exit. Another exemplary dosage form has (i) a semipermeable wall provided around an osmotic formulation a gabapentin-transport moiety complex or a pregabalin-transport moiety complex, an osmagent, and an osmopolymer; and (ii) an exit.

In one embodiment, the dosage form provides a total daily dose of between 200-3600 mg.

In another aspect, the invention provides an improvement in a dosage form comprising gabapentin or pregabalin. The improvement includes a dosage form comprising a complex of gabapentin or pregabalin and a transport moiety associated by a tight-ion pair bond.

In another aspect, the invention includes a method for administering gabapentin or pregabalin, comprising, administering the substance described above to a patient in need thereof.

In one embodiment, the substance is orally administered.

In another aspect, the invention includes a method of preparing a complex of gabapentin or pregabalin and a transport moiety, comprising providing gabapentin or pregabalin; providing a transport moiety; combining the gabapentin or pregabalin and the transport moiety in the presence of a solvent having a dielectric constant less than that of water; whereby the combining results in formation of a complex of gabapentin or pregabalin and the transport moiety.

In one embodiment, combining includes (i) combining the gabapentin or pregabalin and the transport moiety in an aqueous solvent, (ii) adding a solvent having a dielectric constant less than that of water to the aqueous solvent, and (iii) recovering the complex from the solvent.

In another embodiment, combining comprises contacting in a solvent having a dielectric constant at least two fold lower than the dielectric constant of water. Exemplary solvents include methanol, ethanol, acetone, benzene, methylene chloride, and carbon tetrachloride.

In another aspect, the invention includes a method of improving gastrointestinal tract absorption of gabapentin or pregabalin, comprising, providing a complex comprised of gabapentin or pregabalin and a transport moiety, the complex characterized by a tight-ion pair bond; and administering the complex to a patient.

In one embodiment, the improved absorption comprises improved lower gastrointestinal absorption.

In another embodiment, the improved absorption comprises improved absorption in the upper gastrointestinal tract.

These aspects, as well as other aspects, features, and advantages of the invention will become more apparent from the following detailed disclosure of the invention and the accompanying claims.

### BRIEF DESCRIPTION OF THE FIGURES

The following figures are not drawn to scale, and are set forth to illustrate various embodiments of the invention.

FIG. 1 is a diagram of epithelial cells of the gastrointestinal tract, illustrating the transcellular pathway and the paracellular pathway for transport of molecules through the epithelium;

FIG. 2A shows the chemical structure of gabapentin;

FIG. 2B shows the chemical structure of pregabalin;

FIG. 3A shows a generalized synthetic reaction scheme for preparation of a gabapentin-transport moiety or pregabalin-transport moiety complex;

FIG. 3B shows a generalized synthetic reaction scheme for preparation of a gabapentin-transport moiety or pregabalin-transport moiety complex, where the transport moiety includes a sulfate group;

FIG. 3C shows a synthetic reaction scheme for preparation of a gabapentin-alkyl sulfate complex;

FIG. 3D shows a synthetic reaction scheme for preparation of a pregabalin-alkyl sulfate complex;

FIGS. 4A-4D are FTIR scans of gabapentin (FIG. 4A), sodium lauryl sulfate (FIG. 4B), a physical mixture (loose ionic pair) of gabapentin and sodium lauryl sulfate (FIG. 4C), and gabapentin-lauryl sulfate complex (FIG. 4D);

FIG. 5 shows the gabapentin plasma concentration, in ng/mL, in rats as a function of time, in hours, for gabapentin administered intravenously (triangles) and via intubation into a ligated colon (circles) and for a gabapentin lauryl sulfate complex (diamonds) administered via intubation into a ligated colon;

FIG. 6A shows the gabapentin plasma concentration, in ng/mL, in rats as a function of time, in hours, for gabapentin administered intravenously (triangles) and to the duodenum at dosages of 5 mg (circles), 10 mg (squares) and 20 mg (diamonds);

FIG. 6B shows the gabapentin plasma concentration, in ng/mL, in rats as a function of time, in hours, after administration of gabapentin lauryl sulfate complex intravenously (triangles) and to the duodenum at dosages of 5 mg (circles), 10 mg (squares) and 20 mg (diamonds);

FIG. 6C is a plot of gabapentin bioavailability, in percent, as a function of dose following administration of gabapentin (inverted triangles) or of gabapentin lauryl sulfate complex (circles) to the duodenum of rats;

FIG. 7 illustrates an exemplary osmotic dosage form shown in cutaway view,

FIG. 8 illustrates another exemplary osmotic dosage form for a once daily dosing of gabapentin, the dosage form comprising a gabapentin-transport moiety complex or a pregabalin-transport moiety complex, with an optional loading dose of the complex in the outer coating;

FIG. 9 illustrates one embodiment of a once daily gabapentin (or pregabalin) dosage form comprising both gabapentin (or pregabalin) and a gabapentin (or pregabalin)-transport moiety complex, with an optional loading dose of gabapentin (or pregabalin) by coating;

FIGS. 10A-10C illustrate an embodiment of a dosage prior to administration to a subject and comprising a complex of gabapentin (or pregabalin)-transport moiety complex in a matrix (FIG. 10A), in operation after ingestion into the gastrointestinal tract (FIG. 10B), and after sufficient erosion of the matrix has caused separation of the banded sections of the device (FIG. 10C).

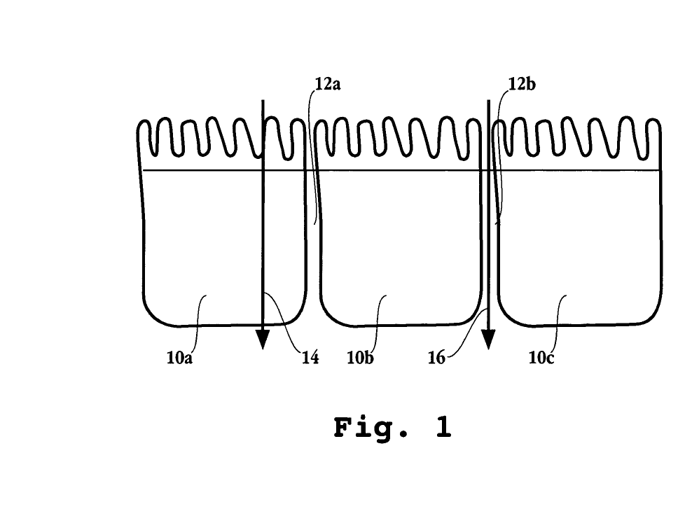

### DETAILED DESCRIPTION

### I. DEFINITIONS

The present invention is best understood by reference to the following definitions, the drawings and exemplary disclosure provided herein.

By “composition” is meant one or more of the gapapein-transport moiety or pregabalin-transport moiety complexes, optionally in combination with additional active pharmaceutical ingredients, and/or optionally in combination with inactive ingredients, such as pharmaceutically-acceptable carriers, excipients, suspension agents, surfactants, disintegrants, binders, diluents, lubricants, stabilizers, antioxidants, osmotic agents, colorants, plasticizers, and the like.

By “complex” is meant a substance comprising a drug moiety and a transport moiety associated by a tight-ion pair bond. A drug-moiety-transport moiety complex can be distinguished from a loose ion pair of the drug moiety and the transport moiety by a difference in octanol/water partitioning behavior, characterized by the following relationship:
Δ Log D=Log D(complex)−Log D(loose-ion pair)≧0.15 (Equation 1)
wherein, D, the distribution coefficient (apparent partition coefficient), is the ratio of the equilibrium concentrations of all species of the drug moiety and the transport moiety in octanol to the same species in water (deionized water) at a set pH (typically about pH=5.0 to about pH=7.0) and at 25 degrees Celsius. Log D (complex) is determined for a complex of the drug moiety and transport moiety prepared according to the teachings herein. Log D (loose-ion pair) is determined for a physical mixture of the drug moiety and the transport moiety in deionized water. For instance, the octanoli/water apparent partition coefficient (D=C_{octanol}/C_{water}) of a putative complex (in deionized water at 25 degree Celsuis) can be determined and compared to a 1:1 (mol/mol) physical mixture of the transport moiety and the drug moiety in deionized water at 25 degree Celsuis. If the difference between the Log D for the putative complex (D+T−) and the Log D for the 1:1 (mol/mol) physical mixture, D⁺∥T⁻ is determined is greater than or equal to 0.15, the putative complex is confirmed as being a complex according to the invention. In preferable embodiments, Δ Log D≧0.20, and more preferably Δ Log D≧0.25, more preferably still Δ Log D≧0.35.

By “dosage form” is meant a pharmaceutical composition in a medium, carrier, vehicle, or device suitable for administration to a patient in need thereof.

By “drug” or “drug moiety” is meant a drug, compound, or agent, or a residue of such a drug, compound, or agent that provides some pharmacological effect when administered to a subject. For use in forming a complex, the drug comprises a(n) acidic, basic, or zwitterionic structural element, or a(n) acidic, basic, or zwitterionic residual structural element.

By “fatty acid” is meant any of the group of organic acids of the general formula CH₃(CₙHₓ)COOH where the hydrocarbon chain is either saturated (x=2n, e.g. palmitic acid, C₁₅H₃₁COOH) or unsaturated (x=2n-2, e.g. oleic acid, CH₃C₁₆H₃₀COOH).

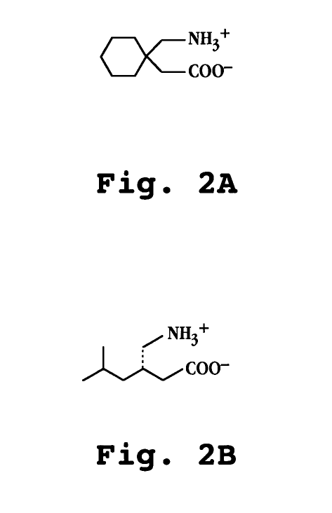

“Gabapentin” refers to 1-(aminomethyl)cyclohexaneacetic acid with a molecular formula of C₉H₁₇NO₂ and a molecular weight of 171.24. It is commercially available under the tradename Neurontin®. Its structure is shown in FIG. 2A.

By “intestine” or “gastrointestinal (G.I.) tract” is meant the portion of the digestive tract that extends from the lower opening of the stomach to the anus, composed of the small intestine (duodenum, jejunum, and ileum) and the large intestine (ascending colon, transverse colon, descending colon, sigmoid colon, and rectum).

By “loose ion-pair” is meant a pair of ions that are, at physiologic pH and in an aqueous environment, are readily interchangeable with other loosely paired or free ions that may be present in the environment of the loose ion pair. Loose ion-pairs can be found experimentally by noting interchange of a member of a loose ion-pair with another ion, at physiologic pH and in an aqueous environment, using isotopic labeling and NMR or mass spectroscopy. Loose ion-pairs also can be found experimentally by noting separation of the ion-pair, at physiologic pH and in an aqueous environment, using reverse phase HPLC. Loose ion-pairs may also be referred to as “physical mixtures,” and are formed by physically mixing the ion-pair together in a medium.

By “lower gastrointestinal tract” or “lower G.I. tract” is meant the large intestine.

By “patient” is meant an animal, preferably a mammal, more preferably a human, in need of therapeutic intervention.

By “tight-ion pair” is meant a pair of ions that are, at physiologic pH and in an aqueous environment are not readily interchangeable with other loosely paired or free ions that may be present in the environment of the tight-ion pair. A tight-ion pair can be experimentally detected by noting the absence of interchange of a member of a tight ion-pair with another ion, at physiologic pH and in an aqueous environment, using isotopic labeling and NMR or mass spectroscopy. Tight ion pairs also can be found experimentally by noting the lack of separation of the ion-pair, at physiologic pH and in an aqueous environment, using reverse phase HPLC.

By “transport moiety” is meant a compound that is capable of forming, or a residue of that compound that has formed, a complex with a drug, wherein the transport moiety serves to improve transport of the drug across epithelial tissue, compared to that of the uncomplexed drug. The transport moiety comprises a hydrophobic portion and a(n) acidic, basic, or zwitterionic structural element, or a(n) acidic, basic, or zwitterionic residual structural element. In a preferred embodiment, the hydrophobic portion comprises a hydrocarbon chain. In an embodiment, the pKa of a basic structural element or basic residual structural element is greater than about 7.0, preferably greater than about 8.0.

By “pharmaceutical composition” is meant a composition suitable for administration to a patient in need thereof.

Pregabalin refers to (S)-(+)-3-(aminomethyl)-5-methylhexanoic acid). Pregabalin is also referred to in the literature as (S)-3-isobutyl GABA or Cl-1008. The structure of pregabalin is shown in FIG. 2B.

By “structural element” is meant a chemical group that (i) is part of a larger molecule, and (ii) possesses distinguishable chemical functionality. For example, an acidic group or a basic group on a compound is a structural element.

By “substance” is meant a chemical entity having specific characteristics.

By “residual structural element” is meant a structural element that is modified by interaction or reaction with another compound, chemical group, ion, atom, or the like. For example, a carboxyl structural element (COOH) interacts with sodium to form a sodium-carboxylate salt, the COO— being a residual structural element.

By “upper gastrointestinal tract” or “upper G.I. tract” is meant that portion of the gastrointestinal tract including the stomach and the small intestine.

### II. COMPLEX FORMATION AND CHARACTERIZATION

As noted above, gabapentin is effective both as an anti-convulsant and in reducing neuropathic pain. Gabapentin, shown in FIG. 2A, is a zwitterionic compound with a pKa₁ of 3.7 and a pKa₂ of 10.7. It is freely soluble in water and in both basic and acidic aqueous solutions. The log of the partition coefficient (n-octanol/0.05M phosphate buffer) at pH 7.4 is −1.25. These properties, along with the fact that it is adsorbed by the L-amino acid transport system, discussed above, results in poor G.I. absorption of the compound. The pH gradient in the G.I. tract ranging from a pH of about 1.2 in the stomach to a pH of about 7.5 in the distal ileum and large intestine (Evans, D. F. et al., Gut, 29:1035-1041 (1988)) means that gabapentin is charged over the range of pH in the G.I. tract, also a contributing factor to its poor absorption. Pregabalin, shown in FIG. 2B, is a structural analog of gabapentin and suffers from some of the same characteristics that result in poor absorption in the lower G.I tract.

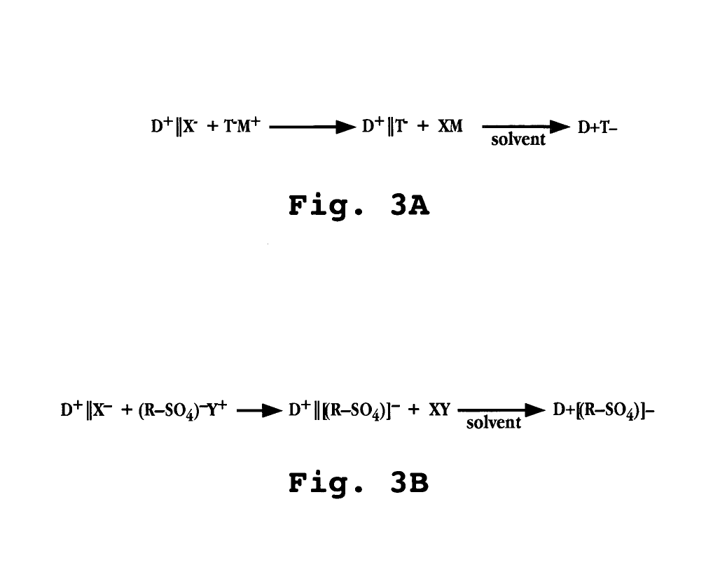

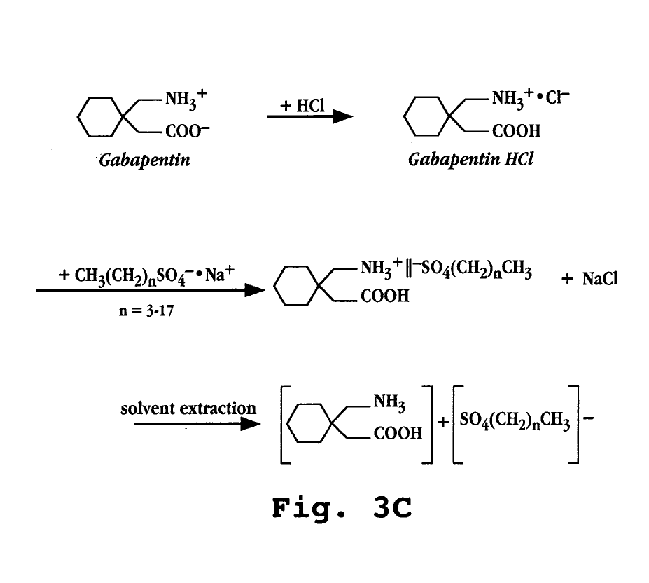

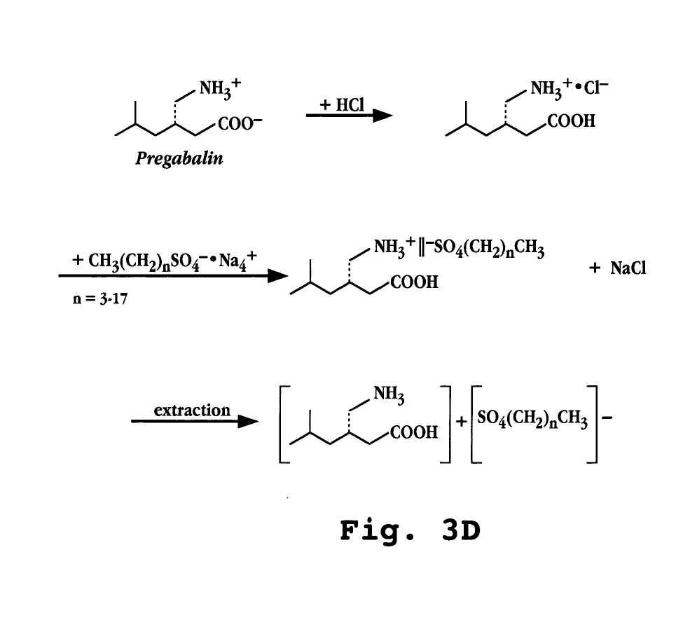

Accordingly, in one aspect, the invention provides a compound comprising gabapentin or pregabalin that has significantly improved lower G.I. tract absorption. The compound is a complex of gabapentin and a transport moiety, or a complex of pregabalin and a transport moiety. The compound can be prepared from a salt of the drug, such as gabapentin hydrochloride or pregabalin hydrochloride, according to the generalized synthetic reaction scheme shown in FIG. 3A. Briefly, the drug in salt form, denoted D+X− in FIG. 3A, is combined with a transport moiety, represented as T⁻ M⁺ in the drawing. Exemplary transport moieties are listed above and include fatty acids, fatty acid salts, alkyl sulfates, benzenesulfonic acid, benzoic acid, fumaric acid, and salicylic acid. The two species are combined in water to form a loose ionic pair (denoted in the figure is D+∥X−)and then solvated in a solvent that has a dielectric constant less than water, as will be discussed below. The process results in formation of a gabapentin-transport moiety complex or a pregabalin-transport moiety complex, where the species in the complex are associated a tight ion pair bond, as denoted in FIG. 3A by the representation D+T−.

FIG. 3B illustrates a more specific synthetic reaction scheme for formation of a gabapentin (or pregabalin)-transport moiety complex. In this scheme, the transport moiety is represented as a salt of an alkyl sulfate, (R—SO₄)⁻ (Y)⁺. The alkyl sulfate salt is mixed with the drug salt in water to form a loose ion pair, denoted in FIG. 3B as D+∥[(R—SO₄)]⁻. An organic solvent having a dielectric constant less than water is added to the aqueous solution of the loose ion pair and the drug-transport moiety complex is extracted, where the drug and the transport moiety are associated by a tight ion pair bond, denoted in the drawing as D+[(R—SO₄)]−.

A specific example of a procedure for preparing a gabapentin-transport moiety complex, where the transport moiety is an alkyl sulfate and more specifically an alkyl sulfate salt, is provided in Example 1A, and illustrated in FIG. 3C. A salt form of gabapentin is prepared, for example, gabapentin HCl, by combining gabapentin with hydrochloric acid. It will be appreciated that other salts of gabapentin can be formed. Then, an alkyl sulfate, such as lauryl sulfate, is added. In Example 1A, the sodium salt of lauryl sulfate was used, however other salts are suitable, such potassium alkyl sulfate or magnesium alkyl sulfate. The gabapentin HCl and the sodium lauryl sulfate are combined to form an ionic pair of gabapentin and lauryl sulfate, denoted in FIG. 3C as a loose ionic pairing between the species. A solvent having a dielectric constant less than water is added to the solution containing the gabapentin and lauryl sulfate and thoroughly mixed and allowed to settle. A gabapentin lauryl sulfate complex is extracted from the solvent phase (non-aqueous phase), typically using a suitable technique to remove a solvent, including but not limited to evaporation, distillation, etc.

In Example 1A, a complex was formed using an alkyl sulfate, lauryl sulfate, as an exemplary transport moiety. It will be understood that lauryl sulfate is merely exemplary and that the preparation procedure is equally applicable to other species suitable as a transport moiety, and to alky sulfates and fatty acids of any carbon chain length. For example, complex formation of gabapentin (or pregabalin) with various alkyl sulfates or fatty acids or salts of the same, where the alkyl chain in the alkyl sulfate or the fatty acid has from 6 to 18 carbon atoms, more preferably 8 to 16 carbon atoms and even more preferably 10 to 14 carbon atoms. The alkyl chain can be saturated or unsaturated. Exemplary saturated alkyl chains in fatty acids contemplated for use in preparation of the complex include butanoic (butyric, 4C); pentanoic (valeric, 5C); hexanoic (caproic, 6C); octanoic (caprylic, 8C); nonanoic (pelargonic, 9C); decanoic (capric, 10C); dodecanoic (lauric, 12C); tetradecanoic (myristic, 14C); hexadecanoic (palmitic, 16C); heptadecanoic (margaric, 17C); and octadecanoic (stearic, 18C); where the systematic name is followed in parenthesis by the fatty acid trivial name and the number of carbon atoms in the fatty acid. Unsaturated fatty acids include oleic acid, linoleic acid, and linolenic acid, all having 18 carbon atoms. Linoleic acid and linolenic acid are polyunsaturated. Exemplary complexes with gabapentin include gabapentin palmitate, gabapentin oleate, gabapentin caprate, gabapentin laurate, gabapentin-lauryl sulfate, gabapentin-decyl sulfate, and gabapentin-tetradecyl sulfate.

Exemplary alkyl sulfates and salts of alkyl sulfates (e.g., sodium, potassium, magnesium, etc), have from 6 to 18 carbon atoms, more preferably 8 to 16 and even more preferably 10 to 14 carbon atoms. Preferred alkyl sulfates include capryl sulfate, lauryl sulfate, and myristyl sulfate. Complex formation of gabapentin or pregabalin with the benzenesulfonic acid, benzoic acid, fumaric acid, and salicylic acid, or the salts of these acids, is also contemplated.

Gabapentin and pregabalin are zwitterionic compounds, permitting the possibility of interaction with positively and negatively charged group. In one embodiment, a transport moiety capable of interaction the positively charged NH₃⁺ moiety of gabapentin and pregabalin is selected, as was discussed with respect to FIGS. 3A-3C. Fatty acids and their salts, alkyl sulfates (either saturated or unsaturated) and their salts (including particularly sodium octyl sulfate, sodium decyl sulfate, sodium lauryl sulfate, and sodium tetradecyl sulfate), benzene sulfonic acid and its salt, benzoic acid and its salt, fumaric acid and its salt, salicylic acid and its salt, or other pharmaceutically acceptable compounds containing at least one carboxylic group and their salts complex with the positively charged group of gabapentin or of pregabalin.

In an alternative embodiment, a transport moiety capable of interaction with the negatively charged COO⁻ group of gabapentin or pregabalin is selected. For example, primary aliphatic amines (both saturated and unsaturated), diethanolamine, ethylenediamine, procaine, choline, tromethamine, meglumine, magnesium, aluminum, calcium, zinc, alkyltrimethylammonium hydroxides, alkyltrimethylammonium bromides, benzalkonium chloride and benzethonium chloride can be used to complex with the negatively charged group of gabapentin and pregabalin.

With continuing reference to Example 1A, the complex comprised of gabapentin-lauryl sulfate was prepared from methylene chloride (chloforom). Methylene chloride is merely an exemplary solvent, and other solvents in which the transport moiety and the drug are soluble are suitable. For example, fatty acids are soluble in chloroform, benzene, cyclohexane, ethanol (95%), acetic acid, and methanol. The solubility (in g/L) of capric acid, lauric acid, myristic acid, palmitic acid, and stearic acid in these solvents is indicated in Table 1.

TABLE 1
Solubility (g/L) of Fatty Acids at 20° C.

| Fatty Acid | | | | | | | | |
|---|---|---|---|---|---|---|---|---|
| (no. | | | | | Ethanol | Acetic | | |
| carbons) | Chloroform | Benzene | Cyclohexanne | Acetone | 95% | acid | Methanol | Acetonitrile |
| capric (10) | 3260 | 3980 | 3420 | 4070 | 4400 | 5670 | 5100 | 660 |
| lauric (12) | 830 | 936 | 680 | 605 | 912 | 818 | 1200 | 76 |
| myristic | 325 | 292 | 215 | 159 | 189 | 102 | 173 | 18 |
| (14) | | | | | | | | |
| palmitic | 151 | 73 | 65 | 53.8 | 49.3 | 21.4 | 37 | 4 |
| (16) | | | | | | | | |
| stearic | 60 | 24.6 | 24 | 15.4 | 11.3 | 1.2 | 1 | <1 |
| (18) | | | | | | | | |

In one embodiment, the solvent used for formation of the complex is a solvent having a dielectric constant less than water, and preferably at least two fold lower than the dielectric constant of water, more preferably at least three-fold lower than that of water. The dielectric constant is a measure of the polarity of a solvent and dielectric constants for exemplary solvents are shown in Table 2.

TABLE 2
Characteristics of Exemplary Solvents

| Solvent | Boiling Pt., ° C. | Dielectric constant |
|---|---|---|
| Water | 100 | 80 |
| Methanol | 68 | 33 |
| Ethanol | 78 | 24.3 |
| 1-propanol | 97 | 20.1 |
| 1-butanol | 118 | 17.8 |
| acetic acid | 118 | 6.15 |
| Acetone | 56 | 20.7 |
| methyl ethyl ketone | 80 | 18.5 |
| ethyl acetate | 78 | 6.02 |
| Acetonitrile | 81 | 36.6 |
| N,N-dimethylformamide (DMF) | 153 | 38.3 |
| dimethyl sulfoxide (DMSO) | 189 | 47.2 |
| Hexane | 69 | 2.02 |
| Benzene | 80 | 2.28 |
| diethyl ether | 35 | 4.34 |
| tetrahydrofuran (THF) | 66 | 7.52 |
| methylene chloride | 40 | 9.08 |
| carbon tetrachloride | 76 | 2.24 |

The solvents water, methanol, ethanol, 1-propanol, 1-butanol, and acetic acid are polar protic solvents having a hydrogen atom attached to an electronegative atom, typically oxygen. The solvents acetone, ethyl acetate, methyl ethyl ketone, and acetonitrile are dipolar aprotic solvents, and are in one embodiment, preferred for use in forming the gabapentin (or pregabalin)-transport moiety complex. Dipolar aprotic solvents do not contain an OH bond but typically have a large bond dipole by virtue of a multiple bond between carbon and either oxygen or nitrogen. Most dipolar aprotic solvents contain a C—O double bond. The dipolar aprotic solvents noted in Table 2 have a dielectric constant at least two-fold lower than water.

FIG. 3D shows a synthetic reaction scheme for formation of a pregabalin lauryl sulfate complex. As described in Example 1B, a salt form of prebgabalin is prepared, for example, pregabalin HCl, by mixing pregabalin with an aqueous solution of hydrochloric acid. It will be appreciated that other salts of pregabalin can be formed. Then, an alkyl sulfate, such as lauryl sulfate, is added. FIG. 3D shows a sodium salt of lauryl sulfate, however other salts are suitable, such potassium alkyl sulfate or magnesium alkyl sulfate. The pregabalin HCl and the sodium lauryl sulfate are mixed to form an ionic pair of pregabalin and lauryl sulfate, denoted in FIG. 3D as a loose ionic pairing between the species. A solvent having a dielectric constant less than water is added to the solution containing the ionic pair of pregabalin and lauryl sulfate and thoroughly mixed and allowed to settle. A pregabalin-lauryl sulfate complex is extracted from the solvent phase (non-aqueous phase), typically using a suitable technique to remove the solvent, including but not limited to evaporation, distillation, etc.

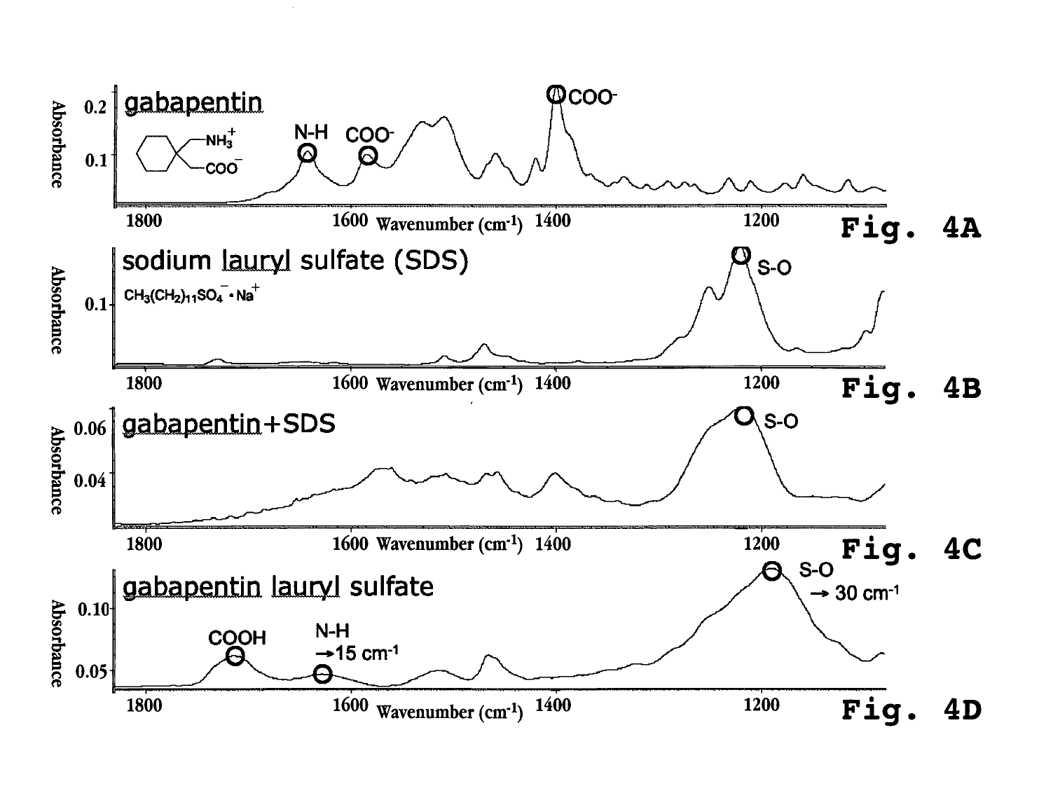

Fourier Transform Infrared Spectroscopy (FTIR) was use to analyze the gabapentin-lauryl sulfate complex formed as described in Example 1A. The FTIR/ATR methodology is described in the methods section below. For comparison, FTIR/ATR spectra of gabapentin, sodium lauryl sulfate, and of a 1:1 molar ratio physical mixture of gabapentin and sodium lauryl sulfate (two components were dissolved in methanol and dried in air as a solid film) were also generated, and the results are shown in FIGS. 4A-4D. The spectrum for gabapentin is shown in FIG. 4A, and the peaks corresponding to the NH and COO moieties are indicated. The spectrum for sodium lauryl sulfate is shown in FIG. 4B, and a main, doublet peak corresponding to the S—O moiety is observed between 1300-1200 cm⁻¹. A 1:1 molar mixture of gabapentin HCl and sodium lauryl sulfate in water is shown in FIG. 4C, and an attenuation of the distinct pattern characteristic of gabapentin is apparent and a broadening of the S—O peak (1300-1200 cm⁻¹) from the sodium lauryl sulfate observed. FIG. 4D shows the FTIR spectrum for the complex formed by the procedure in Example 1A, where two peaks corresponding to the COO— group of gabapentin disappeared and were replaced by a peak of COOH group in gabapentin lauryl sulfate complex, indicating the charge blocking of COO—. Deformation of N-H moiety of gabapentin was observed by the 15 cm⁻¹ shift in the spectra of gabpentin lauryl sulfate. This shift of bands for N—H bond indicates the protonation of the N—H groups in the resulting complex. The peak at 1250 cm⁻¹ that is indicative of the S—O absorption in the spectra of sodium lauryl sulfate was shifted 30 cm⁻¹ as shown in the spectra of gabapentin complex, suggesting the interaction of gabapentin with sulfate group of sodium lauryl sulfate. The FTIR scans showed that the complex formed of gabapentin is different from the physical mixture of two components.

While not wishing to be bound by specific understanding of mechanisms, the inventors reason as follows. When loose ion-pairs are placed in a polar solvent environment, it is assumed that polar solvent molecules will insert themselves in the space occupied by the ionic bond, thus driving apart the bound ions. A salvation shell, comprising polar solvent molecules electrostatically bonded to a free ion, may be formed around the free ion. This solvation shell then prevents the free ion from forming anything but a loose ion-pairing ionic bond with another free ion. In a situation wherein there are multiple types of counter ions present in the polar solvent, any given loose ion-pairing may be relatively susceptible to counter-ion competition.

This effect is more pronounced as the polarity, expressed as the dielectric constant of the solvent, increases. Based on Coulomb's law, the force between two ions with charges (q1) and (q2) and separated by a distance (r) in a medium of dielectric constant (e) is:

$$F = - \frac{{q}_{1} {q}_{2}}{4 π {ɛ}_{0} ɛ {r}^{2}} \qquad ( Equation 2 )$$

where ε₀ is the constant of permittivity of space. The equation shows the importance of dielctric constant (ε) on the stability of a loose ion-pair in solution. In aqueous solution that has a high dielectric constant (ε=80), the electrostatic attraction force is significantly reduced if water molecules attack the ionic bonding and separate the opposite charged ions.

Therefore, high dielectric constant solvent molecules, once present in the vicinity of the ionic bond, will attack the bond and eventually break it. The unbound ions then are free to move around in the solvent. These properties define a loose ion-pair.

Tight ion-pairs are formed differently from loose-ion pairs, and consequently poses different properties from a loose ion-pair. Tight ion-pairs are formed by reducing the number of polar solvent molecules in the bond space between two ions. This allows the ions to move tightly together, and results in a bond that is significantly stronger than a loose ion-pair bond, but is still considered an ionic bond. As disclosed more fully herein, tight ion-pairs are obtained using less polar solvents than water so as to reduce entrapment of polar solvents between the ions.

For additional discussion of loose and tight ion-pairs, D. Quintanar-Guerrero et al., Pharm. Res., 14(2):119-127 (1997).

The difference between loose and tight ion-pairing also can be observed using chromatographic methods. Using reverse phase chromatography, loose ion-pairs can be readily separated under conditions that will not separate tight ion-pairs.

Bonds according to this invention may also be made stronger by selecting the strength of the cation and anion relative to one another. For instance, in the case where the solvent is water, the cation (base) and anion (acid) can be selected to attract one another more strongly. If a weaker bond is desired, then weaker attraction may be selected.

Portions of biological membranes can be modeled to a first order approximation as lipid bilayers for purposes of understanding molecular transport across such membranes. Transport across the lipid bilayer portions (as opposed to active transporters, etc.) is unfavorable for ions because of unfavorable portioning. Various researchers have proposed that charge neutralization of such ions can enhance cross-membrane transport.

In the “ion-pair” theory, ionic drug moieties are paired with transport moiety counter ions to “bury” the charge and render the resulting ion-pair more liable to move through a lipid bilayer. This approach has generated a fair amount of attention and research, especially with regards to enhancing absorption of orally administered drugs across the intestinal epithelium.

While ion-pairing has generated a lot of attention and research, it has not always generated a lot of success. For instance, ion-pairs of two antiviral compounds were found not to result in increased absorption due to the effects of the ion-pair on trans-cellular transport, but rather to an effect on monolayer integrity (J. Van Gelder et al., Int. J. of Pharmaceutics, 186:127-136 (1999). The authors concluded that the formation of ion pairs may not be very efficient as a strategy to enhance trans-epithelial transport of charged hydrophilic compounds as competition by other ions found in in vivo systems may abolish the beneficial effect of counter-ions. Other authors have noted that absorption experiments with ion-pairs have not always pointed at clear-cut mechanisms (D. Quintanar-Guerrero et al., Pharm. Res., 14(2):119-127 (1997)).

The inventors have unexpectedly discovered that a problem with these ion-pair absorption experiments is that they were performed using loose-ion pairs, rather than tight ion-pairs. Indeed, many ion-pair absorption experiments disclosed in the art do not even expressly differentiate between loose ion-pairs and tight ion-pairs. One of skill has to distinguish that loose ion-pairs are disclosed by actually reviewing the disclosed methods of making the ion-pairs and noting that such disclosed methods of making are directed to loose ion-pairs not tight ion-pairs. Loose ion-pairs are relatively susceptible to counter-ion competition, and to solvent-mediated (e.g. water-mediated) cleavage of the ionic bonds that bind loose ion-pairs. Accordingly, when the drug moiety of the ion-pair arrives at an intestinal epithelial cell membrane wall, it may or may not be associated in a loose ion-pair with a transport moiety. The chances of the ion-pair existing near the membrane wall may depend more on the local concentration of the two individual ions than on the ion bond keeping the ions together. Absent the two moieties being bound when they approached an intestinal epithelial cell membrane wall, the rate of absorption of the non-complexed drug moiety might be unaffected by the non-complexed transport moiety. Therefore, loose ion-pairs might have only a limited impact on absorption compared to administration of the drug moiety alone.

In contrast, the inventive complexes possess bonds that are more stable in the presence of polar solvents such as water. Accordingly, the inventors reasoned that, by forming a complex, the drug moiety and the transport moiety would be more likely to be associated as ion-pairs at the time that the moieties would be near the membrane wall. This association would increase the chances that the charges of the moieties would be buried and render the resulting ion-pair more liable to move through the cell membrane.

In an embodiment, the complex comprises a tight ion-pair bond between the drug moiety and the transport moiety. As discussed herein, tight ion-pair bonds are more stable than loose ion-pair bonds, thus increasing the likelihood that the drug moiety and the transport moiety would be associated as ion-pairs at the time that the moieties would be near the membrane wall. This association would increase the chances that the charges of the moieties would be buried and render the tight ion-pair bound complex more liable to move through the cell membrane.

It should be noted that the inventive complexes may improve absorption relative to the non-complexed drug moiety throughout the G.I. tract, not just the lower G.I. tract, as the complex is intended to improve transcellular transport generally, not just in the lower G.I. tract. For instance, if the drug moiety is a substrate for an active transporter found primarily in the upper G.I., the complex formed from the drug moiety may still be a substrate for that transporter. Accordingly, the total transport may be a sum of the transport flux effected by the transporter plus the improved transcellular transport provided by the present invention. In an embodiment, the inventive complex provides improved absorption in the upper G.I. tract, the lower G.I. tract, and both the upper G.I. tract and the lower G.I. tract.

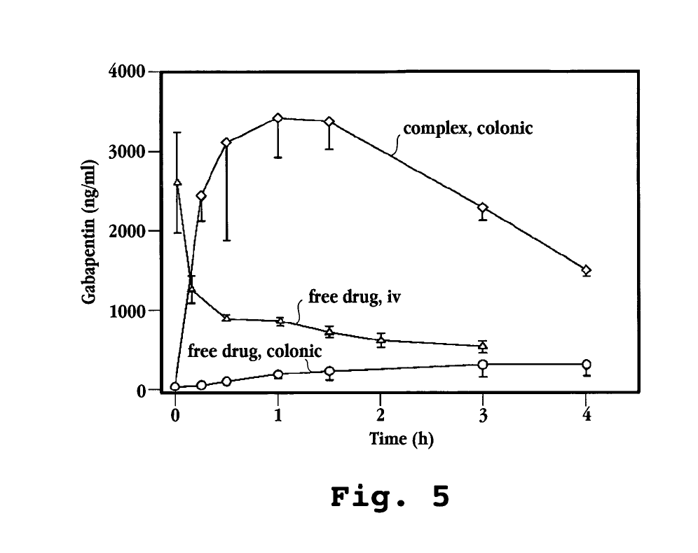

In a study conducted in support of the invention, the lower G.I. absorption of the gabapentin-lauryl sulfate complex was characterized in vivo using a flush ligated colonic model in rats. As described in Example 2, a 10 mg/rat dose of gabapentin in the form of gabapentin-lauryl sulfate complex or as neat gabapentin was intubated into the ligated colon of test rats (n=3 in each group). A third group of rats (n=3) was given 1 mg of gabapentin intravenously. Blood samples were withdrawn periodically for analysis of gabapentin concentration. The data is shown in FIG. 5.

With reference to FIG. 5, gabapentin administered intravenously (triangles) gives a high initial plasma concentration with a sharply decreasing concentration over the first 15 minutes. When gabapentin is administered as an intracolonic bolus (circles) a slow absorption of the drug occurs. In contract, when the drug is administered to the lower G.I. tract in the form of a gabapentin-lauryl sulfate complex (diamonds), a rapid uptake of drug occurs, with a Cmax observed one hour after intubation.

Pharmacokinetic parameters from this study are shown in Table 3. The area under the curve (AUC) is determined from time zero to time infinity based on 1 mg of gabapentin/rat for each of the gabapentin dosages, where time infinity was estimated by assuming a log-linear decline. Gabapentin bioavailability is expressed as a percent of the gabapentin concentration resulting from intravenous administration of the drug.

TABLE 3

| Drug Form | AUC∞ | bioavailability |
|---|---|---|
| (route of administration) | (ng · h/mL-mg) | (%) |
| gabapentin (iv) | 6090.3 | 100 |
| gabapentin (colonic) | 301.4 | 4.9 |
| gabapentin lauryl sulfate | 3854.1 | 63.3 |
| complex (colonic) | | |

The enhanced colonic absorption provided by the complex of gabapentin and lauryl sulfate is apparent from the markedly improved bioavailability of the drug when administered to the lower G.I. tract in the form of the complex relative to the neat drug. The gabapentin-lauryl sulfate complex provided a 13-fold improvement in bioavailability relative to that of the neat drug. Accordingly, the invention contemplates a compound comprised of a complex formed of gabapentin (or pregabalin) and a transport moiety, wherein the complex provides at least a 5-fold increase, more preferably at least a 10-fold increase, and more preferably at least a 12-fold increase in colonic absorption relative to colonic absorption of gabapentin (or pregabalin), as evidenced by gabapentin (or pregabalin) bioavailability determined from gabapentin (or pregabalin) plasma concentration. Thus, gabapentin (or pregabalin) when administered in the form of a gabapentin (or pregabalin)-transport moiety complex provides a significantly enhanced colonic absorption of gabapentin (or pregabalin) into the blood.

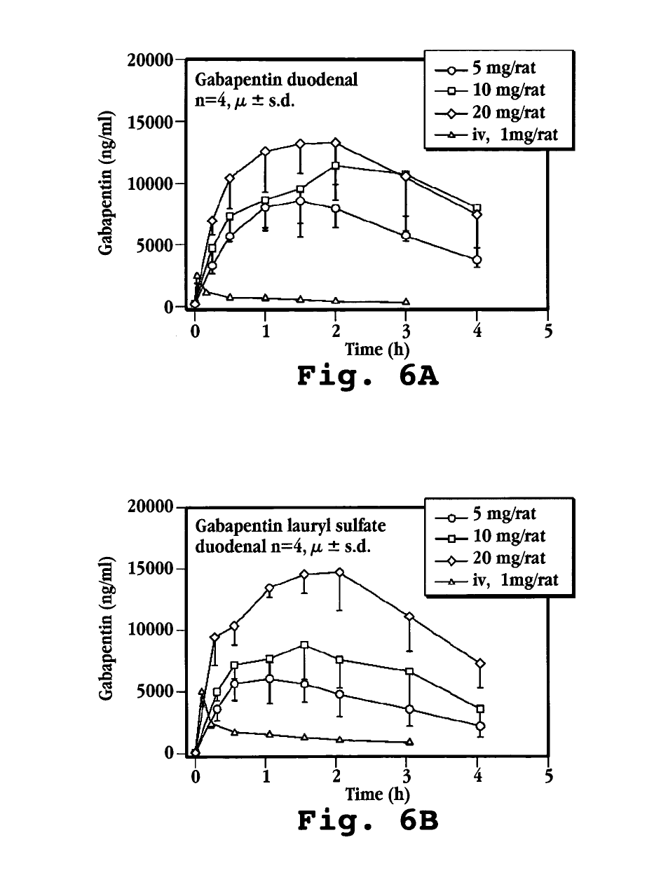

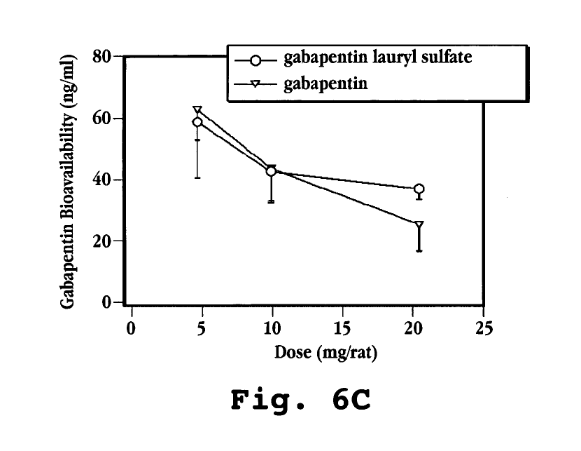

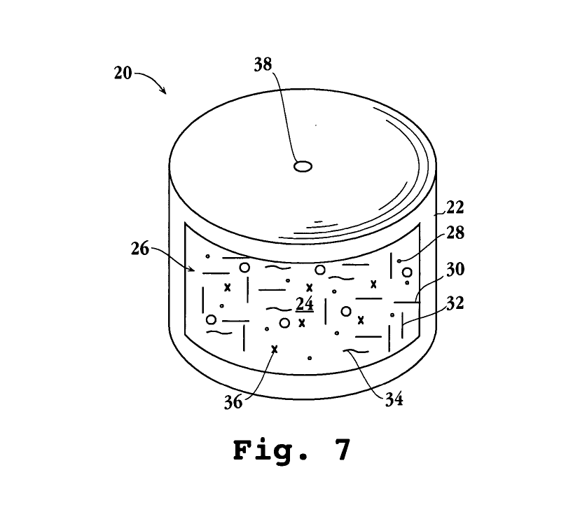

Another study was conducted where gabapentin or gabapentin-lauryl sulfate complex were placed in the duodenum of rats, as described in Example 3. Doses of 5 mg/rat, 10 mg/rat, 20 mg/rat were administered and blood samples taken as a function of time for determination of gabapentin concentration. Another group of test animals received gabapentin or gabapentin-lauryl sulfate complex intravenously. The results are shown in FIGS. 6A-6C.

FIG. 6A shows the gabapentin plasma concentration, in ng/mL, in the animals treated with neat gabapentin, administered intravenously (triangles) and to the duodenum at dosages of 5 mg (circles), 10 mg (squares) and 20 mg (diamonds). An increasing blood concentration with increasing dose was observed for the animals receiving drug via intubation into the duodenum. Naturally, the lower plasma drug concentration for the animals treated intravenously (triangles) is due to the lower drug dose.

FIG. 6B shows the results for the animals receiving gabapentin-lauryl sulfate complex intravenously (triangles) and directly to the duodenum at dosages of 5 mg (circles), 10 mg (squares), and 20 mg (diamonds). While the absolute blood concentrations of the animals receiving gabapentin-lauryl sulfate complex are lower than the animals treated with gabapentin, the data shows that absorption of gabapentin from the complex is enhanced relative to absorption of the neat drug, due perhaps in part to the L-amino acid transport system not being saturated and/or the increased transport via other mechanisms provided by the complex. This is evident from a comparison of the blood concentration between the 5 mg and 10 mg dose and between the 10 mg and 20 mg dose in FIGS. 6A and 6B, where the increase in blood concentration with increased dose is greater for gabapentin administered in the form of the complex.

FIG. 6C shows the percent bioavailability of gabapentin administered as the neat drug (inverted triangles) or as gabapentin lauryl sulfate complex (circles) to the duodenum of rats. Percent bioavailability is determined relative to gabapentin administered intravenously. At a dosage of 20 mg, gabapentin-lauryl sulfate complex exhibited a higher bioavailability than did the neat drug. The increased bioavailability at the higher doses is likely due to the enhanced absorption offered by the complex, where uptake in the G.I. tract is not limited to uptake by the L-amino acid transport system for the complex, but is also occurring by transcellular and paracellular mechanisms.

Table 4 shows the pharmacokinetic analysis from the study, where the area under curve from 0 to 4 hours was determined, and normalized to a 1 mg does of gabapentin/kg rat. The data relating to the hour 4 point for gabapentin (iv) assumes a log-linear decline from the data measured for the first three hours. Percent bioavailability is relative to the bioavailability of intravenously administered gabapentin.

TABLE 4

| | | AUC | |
|---|---|---|---|
| | Dose | (0˜4 h, ng · h/ | Bioavailability |
| Drug Form | (mg/kg. ± s.d.) | ml-mg ± s.d.)* | (%) |
| gabapentin (iv) | 1 | 2727.1 ± 259.1 | 100.0 |
| gabapentin (duodenal) | 14.8 ± 0.1 | 1705.2 ± 257.2 | 62.5 ± 9.4 |
| gabapentin (duodenal) | 30.6 ± 1.7 | 1205.7 ± 276.3 | 44.2 ± 10.1 |
| gabapentin (duodenal) | 59.8 ± 1.7 | 726.1 ± 223.9 | 26.2 ± 8.2 |
| gabapentin lauryl | 14.0 ± 0.1 | 1604.3 ± 479.1 | 58.8 ± 17.6 |
| sulfate(duodenal) | | | |
| gabapentin lauryl | 29.1 ± 1.1 | 1182.2 ± 267.9 | 43.3 ± 9.8 |
| sulfate(duodenal) | | | |
| gabapentin lauryl | 58.1 ± 2.3 | 1033.9 ± 88.9 | 37.9 ± 3.3 |
| sulfate(duodenal) | | | |

*Normalized to dose of 1 mg gabapentin/kg.

The AUC and bioavailability data show that as the dose increases, colonic absorption of gabapentin is improved when the drug is provided in the form of a gapapentin-transport moiety complex.

While the experimental data is based on gabapentin, it will be understood that the findings extend to pregabalin, an analog of gabapentin. Examples 4 and 5 describe methods for determining the in vivo absorption of a pregabalin-lauryl sulfate complex.

### III. EXEMPLARY DOSAGE FORMS AND METHODS OF USE

The complex described above provides an enhanced absorption rate in the G.I. tract, and in particular in the lower G.I. tract. Dosage forms and methods of treatment using the complex and its increased colonic absorption will now be described. It will be appreciated that the dosage forms described below are merely exemplary. It will also be appreciated that the dosage forms are equally applicable to gabapentin, pregabalin, or a mixture thereof. In the discussion below, reference is made to gabapentin; yet it will be understood that the discussion also applies to pregabalin.

A variety of dosage forms are suitable for use with the gabapentin-transport moiety complex. As discussed above, a dosage form that provides once daily dosing to achieve a therapeutic efficacy for at least about 12 hours, more preferably for at least 15 hours, and still more preferably for at least about 20 hours. The dosage form may be configured and formulated according to any design that delivers a desired dose of gabapentin. Typically, the dosage form is orally administrable and is sized and shaped as a conventional tablet or capsule. Orally administrable dosage forms may be manufactured according to one of various different approaches. For example, the dosage form may be manufactured as a diffusion system, such as a reservoir device or matrix device, a dissolution system, such as encapsulated dissolution systems (including, for example, “tiny time pills”, and beads) and matrix dissolution systems, and combination diffusion/dissolution systems and ion-exchange resin systems, as described in Remington's Pharmaceutical Sciences, 18^{th} Ed., pp. 1682-1685 (1990).

A specific example of a dosage form suitable for use with the gabapentin-transport moiety complex is an osmotic dosage form. Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. An advantage to osmotic systems is that their operation is pH-independent and, thus, continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release, 35:1-21 (1995). Osmotic dosage forms are also described in detail in the following U.S. patents, each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

An exemplary dosage form, referred to in the art as an elementary osmotic pump dosage form, is shown in FIG. 11. Dosage form 20, shown in a cutaway view, is also referred to as an elementary osmotic pump, and is comprised of a semi-permeable wall 22 that surrounds and encloses an internal compartment 24. The internal compartment contains a single component layer referred to herein as a drug layer 26, comprising a gabapentin-transport moiety complex 28 in an admixture with selected excipients. The excipients are adapted to provide an osmotic activity gradient for attracting fluid from an external environment through wall 22 and for forming a deliverable gabapentin-transport moiety complex formulation upon imbibition of fluid. The excipients may include a suitable suspending agent, also referred to herein as drug carrier 30, a binder 32, a lubricant 34, and an osmotically active agent referred to as an osmagent 36. Exemplary materials for each of these components are provided below.

Semi-permeable wall 22 of the osmotic dosage form is permeable to the passage of an external fluid, such as water and biological fluids, but is substantially impermeable to the passage of components in the internal compartment. Materials useful for forming the wall are essentially nonerodible and are substantially insoluble in biological fluids during the life of the dosage form. Representative polymers for forming the semi-permeable wall include homopolymers and copolymers, such as, cellulose esters, cellulose ethers, and cellulose ester-ethers. Flux-regulating agents can be admixed with the wall-forming material to modulate the fluid permeability of the wall. For example, agents that produce a marked increase in permeability to fluid such as water are often essentially hydrophilic, while those that produce a marked permeability decrease to water are essentially hydrophobic. Exemplary flux regulating agents include polyhydric alcohols, polyalkylene glycols, polyalkylenediols, polyesters of alkylene glycols, and the like.

In operation, the osmotic gradient across wall 22 due to the presence of osmotically-active agents causes gastric fluid to be imbibed through the wall, swelling of the drug layer, and formation of a deliverable gabapentin-transport moiety complex-containing formulation (e.g., a solution, suspension, slurry or other flowable composition) within the internal compartment. The deliverable gabapentin-transport moiety complex formulation is released through an exit 38 as fluid continues to enter the internal compartment. Even as the complex-containing formulation is released from the dosage form, fluid continues to be drawn into the internal compartment, thereby driving continued release. In this manner, gabapentin-transport moiety complex is released in a sustained and continuous manner over an extended time period.

Preparation of a dosage form like that shown in FIG. 7 is described in Example 6A for gabapentin-transport moiety complex and in Example 6B for a pregabalin-transport moiety complex.

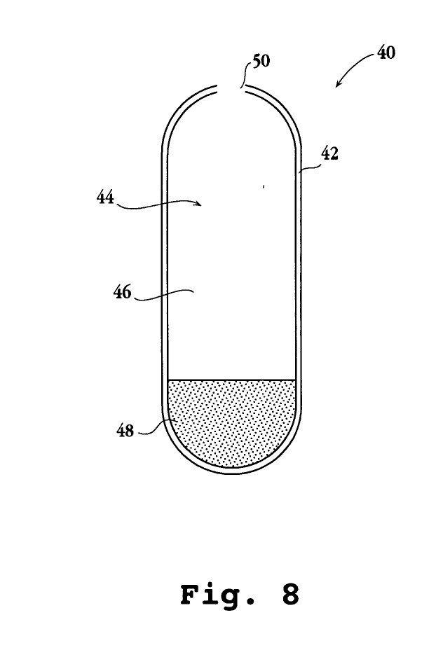

FIG. 8 is a schematic illustration of another exemplary osmotic dosage form. Dosage forms of this type are described in detail in U.S. Pat. Nos. 4,612,008; 5,082,668; and 5,091,190, which are incorporated by reference herein. In brief, dosage form 40, shown in cross-section, has a semi-permeable wall 42 defining an internal compartment 44. Internal compartment 44 contains a bilayered-compressed core having a drug layer 46 and a push layer 48. As will be described below, push layer 48 is a displacement composition that is positioned within the dosage form such that as the push layer expands during use, the materials forming the drug layer are expelled from the dosage form via one or more exit ports, such as exit port 50. The push layer can be positioned in contacting layered arrangement with the drug layer, as illustrated in FIG. 8, or can have one or more intervening layers separating the push layer and drug layer.

Drug layer 46 comprises a gabapentin-transport moiety complex in an admixture with selected excipients, such as those discussed above with reference to FIG. 7. An exemplary dosage form can have a drug layer was comprised of ferrous-laurate complex, a poly(ethylene oxide) as a carrier, sodium chloride as an osmagent, hydroxypropylmethylcellulose as a binder, and magnesium stearate as a lubricant.

Push layer 48 comprises osmotically active component(s), such as one or more polymers that imbibes an aqueous or biological fluid and swells, referred to in the art as an osmopolymer. Osmopolymers are swellable, hydrophilic polymers that interact with water and aqueous biological fluids and swell or expand to a high degree, typically exhibiting a 2-50 fold volume increase. The osmopolymer can be non-crosslinked or crosslinked, and in a preferred embodiment the osmopolymer is at least lightly crosslinked to create a polymer network that is too large and entangled to easily exit the dosage form during use. Examples of polymers that may be used as osmopolymers are provided in the references noted above that describe osmotic dosage forms in detail. A typical osmopolymer is a poly(alkylene oxide), such as poly(ethylene oxide), and a poly(alkali carboxymethylcellulose), where the alkali is sodium, potassium, or lithium. Additional excipients such as a binder, a lubricant, an antioxidant, and a colorant may also be included in the push layer. In use, as fluid is imbibed across the semi-permeable wall, the osmopolymer(s) swell and push against the drug layer to cause release of the drug from the dosage form via the exit port(s).

The push layer can also include a component referred to as a binder, which is typically a cellulose or vinyl polymer, such as poly-n-vinylamide, poly-n-vinylacetamide, poly(vinyl pyrrolidone), poly-n-vinylcaprolactone, poly-n-vinyl-5-methyl-2-pyrrolidone, and the like. The push layer can also include a lubricant, such as sodium stearate or magnesium stearate, and an antioxidant to inhibit the oxidation of ingredients. Representative antioxidants include, but are not limited to, ascorbic acid, ascorbyl palmitate, butylated hydroxyanisole, a mixture of 2 and 3 tertiary-butyl-4-hydroxyanisole, and butylated hydroxytoluene.

An osmagent may also be incorporated into the drug layer and/or the push layer of the osmotic dosage form. Presence of the osmagent establishes an osmotic activity gradient across the semi-permeable wall. Exemplary osmagents include salts, such as sodium chloride, potassium chloride, lithium chloride, etc. and sugars, such as raffinose, sucrose, glucose, lactose, and carbohydrates.

With continuing reference to FIG. 8, the dosage form can optionally include an overcoat (not shown) for color coding the dosage forms according to dose or for providing an immediate release of gabapentin, pregabalin, or other drug.

In use, water flows across the wall and into the push layer and the drug layer. The push layer imbibes fluid and begins to swell and, consequently, pushes on drug layer 44 causing the material in the layer to be expelled through the exit orifice and into the gastrointestinal tract. Push layer 48 is designed to imbibe fluid and continue swelling, thus continually expelling drug from the drug layer throughout the period during which the dosage form is in the gastrointestinal tract. In this way, the dosage form provides a continuous supply of gabapentin-transport moiety complex to the gastrointestinal tract for a period of 15 to 20 hours, or through substantially the entire period of the dosage form's passage through the G.I. tract. Since the gabapentin-transport moiety complex is absorbed in both the upper and lower G.I. tracts, administration of the dosage form provides delivery of gabapentin into the blood stream over period time the dosage form is in transit in the G.I. tract.

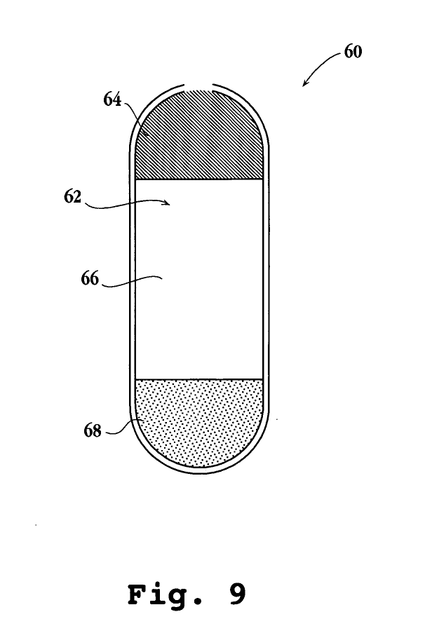

Another exemplary dosage form is shown in FIG. 9. Osmotic dosage form 60 has a tri-layered core 62 comprised of a first layer 64 of gabapentin, a second layer 66 of a gabapentin-transport moiety complex, and a third layer 68 referred to as a push layer. Dosage forms of this type are described in detail in U.S. Pat. Nos. 5,545,413; 5,858,407; 6,368,626, and 5,236,689, which are incorporated by reference herein. As set forth in Example 7, tri-layered dosage forms are prepared to have a first layer of 85.0 wt % gabapentin, 10.0 wt % polyethylene oxide of 100,000 molecular weight, 4.5 wt % polyvinylpyrrolidone having a molecular weight of about 35,000 to 40,000, and 0.5 wt % magnesium stearate. The second layer is comprised 93.0 wt % gabapentin-transport moiety complex (prepared as described in Example 1A), 5.0 wt % polyethylene oxide 5,000,000 molecular weight, 1.0 wt % polyvinylpyrrolidone having molecular weight of about 35,000 to 40,000, and 1.0 wt % magnesium stearate.

The push layer consists of 63.67 wt % of polyethylene oxide, 30.00 wt % sodium chloride, 1.00 wt % ferric oxide, 5.00 wt % hydroxypropylmethylcellulose, 0.08 wt % butylated hydroxytoluene and 0.25 wt % magnesium stearate. The semi-permeable wall is comprised of 80.0 wt % cellulose acetate having a 39.8 % acetyl content and 20.0% polyoxyethylene-polyoxypropylene copolymer.

Dissolution rates of dosage forms, such as those shown in FIGS. 7-9, can be determined according to procedure set forth in Example 8. In general, release of drug formulation from the dosage form begins after contact with an aqueous environment, where, depending on the dosage form, the drug formulation contains gabapentin or gabapentin-transport moiety complex. For example, in the dosage form illustrated in FIG. 7, release of gabapentn-transport moiety complex is released after contact with an aqueous environment and continues for the lifetime of the device. The dosage form illustrated in FIG. 9 provides an initial release of gabapentin, present in the drug layer adjacent the exit orifice, with release of gabapentin-transport moiety complex occurring subsequently.

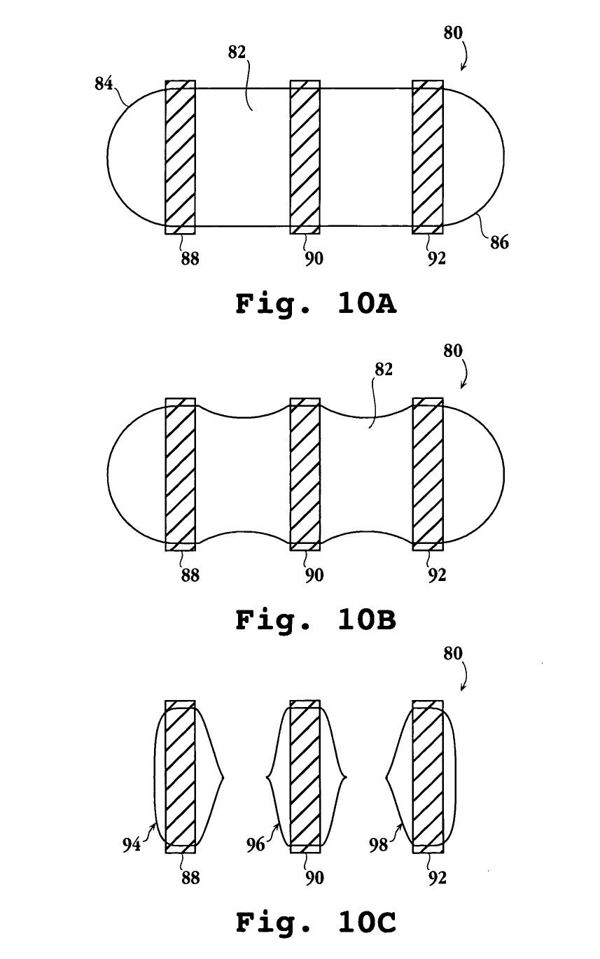

FIGS. 10A-10C illustrate another exemplary dosage form, known in the art and described in U.S. Pat. Nos. 5,534,263; 5,667,804; and 6,020,000, which are specifically incorporated by reference herein. Briefly, a cross-sectional view of a dosage form 80 is shown prior to ingestion into the gastrointestinal tract in FIG. 10A. The dosage form is comprised of a cylindrically shaped matrix 82 comprising a gabapentin-transport moiety complex. Ends 84, 86 of matrix 82 are preferably rounded and convex in shape in order to ensure ease of ingestion. Bands 88, 90, and 92 concentrically surround the cylindrical matrix and are formed of a material that is relatively insoluble in an aqueous environment. Suitable materials are set forth in the patents noted above and in Example 9 below.

After ingestion of dosage form 80, regions of matrix 82 between bands 88, 90, 92 begin to erode, as illustrated in FIG. 10B. Erosion of the matrix initiates release of the gabapentin-transport moiety complex into the fluidic environment of the G.I. tract. As the dosage form continues transit through the G.I. tract, the matrix continues to erode, as illustrated in FIG. 10C. Here, erosion of the matrix has progressed to such an extent that the dosage form breaks into three pieces, 94, 96, 98. Erosion will continue until the matrix portions of each of the pieces have completely eroded. Bands 94, 96, 98 will thereafter be expelled from the G.I. tract.

It will be appreciated the dosage forms described in FIGS. 7-10 are merely exemplary of a variety of dosage forms designed for and capable of achieving delivery of a gabapentin-transport moiety complex to the lower G.I. tract. Those of skill in the pharmaceutical arts can identify other dosage forms that would be suitable.

In another aspect, the invention provides a method for administering gabapentin to a patient by administering a composition or a dosage form that contains a complex of gabapentin and a transport moiety, the complex characterized by a tight-ion pair bond between the gabapentin (or pregabalin) and the transport moiety. A composition comprising the complex and a pharmaceutically-acceptable vehicle are administered to the patient, typically via oral administration.

The dose administered is generally adjusted in accord with the age, weight, and condition of the patient, taking into consideration the dosage form and the desired result. In general, the dosage forms and compositions of the gabapentin-transport moiety complex are administered in amounts recommended for gabapentin (Neurontin®) therapy, as set forth in the Physician's Desk Reference. A typical dose for controlling seizures in epiletic patients is 900-1800 mg per day. Typical doses for use in alleviating neuropathic pain are 600-3600 mg per day (Backonja, M., Clinical Therapies, 23(1) (2003)). It will be appreciated that these dose ranges represent approximate ranges and that the increased absorption provided by the complex will alter the required dose.

With respect to pregabalin, the dose administered will also be adjusted in accord with the age, weight, and condition of the patient, taking into consideration the dosage form and the desired result. In general, a dose of at least about 300 mg day is provided and is increased as needed to provide a reduction in perceived pain relief. Reductions in pain can be measured using numerical pain rating scales, such as the Short-Form McGill Pain Questionnaire (Dworkin, R. H. et al., Neurology, 60:1274 (2003)).

From the foregoing, it can be seen how various objects and features of the invention are met. A complex consisting of gabapentin or pregabalin and a transport moiety, the gabapentin (or pregabalin) and transport moiety associated by a non-covalent, tight-ion pair bond, provides an enhanced G.I. absorption of the drug. The complex is prepared from a novel process, where gabapentin or pregabalin is contacted with a transport moiety, such as an alkyl sulfate or a fatty acid, solubilized in a solvent that is less polar than water, the lower polarity evidenced, for example, by a lower dielectric constant. Contact of the drug with the transport moiety-solvent mixture results in formation of a complex between the drug (gabapentin or pregabalin) and the transport moiety, where the two species are associated by a tight-ion pair bond.

### IV. EXAMPLES

The following examples further illustrate the invention described herein and are in no way intended to limit the scope of the invention.

Methods

1. FTIR: Fouier Transform Infrared Spectroscopy was performed on a Perkin-Elmer Spectrum 2000 spectrometer system equipped with an Attenuated Total Reflectance (ATR) accessory and liquid N₂ cooled MCT (mercury cadmium telluride) detector.

### Example 1

### Preparation of Gabapentin-Transport Moiety Complex and Pregabalin-Transport Moiety Complex

Gabapentin-Transport Moiety Complex
1. A solution of 0.5 mL 36.5% hydrochloric acid (5 mmol HCl) in 25 mL deionized water was prepared.
2. 5 mmol gabapentin (0.86 g) was added to the solution in step 1. The mixture was stirred for 10 min at room temperature. Gabapentin hydrochloride was formed.
3. 5 mmol sodium lauryl sulfate (1.4 g) was added to the aqueous solution in step 2. The mixture was stirred for 20 min at room temperature.
4. 50 mL dichloromethane was added to the solution in step 3. The mixture was stirred for 2 hours at room temperature.
5. The mixture of step 4 was transferred to a separatory funnel and allowed to settle for 3 hours. Two phases were formed, a lower phase of dichloromethane and an upper phase of water.
6. The upper and lower phases in step 5 were separated. The lower dichloromethane phase was recovered and the dichloromethane was evaporated to dryness at room temperature, followed by drying in a vacuum oven for 4 hours at 40° C. A complex of gabapentin-lauryl sulfate (1.9 g) was obtained. Total yield was 87% relative to theoretical amount calculated from the initial amounts of gabapentin and sodium lauryl sulfate.

Pregabalin-Transport Moiety Complex
1. A solution of 0.5 mL 36.5% hydrochloric acid (5 mmol HCl) in 25 mL deionized water is prepared.
2. 5 mmol pregabalin (0.80 g) is added to the solution in step 1. The mixture is stirred for 10 min at room temperature. Pregabalin hydrochloride is formed.
3. 5 mmol sodium lauryl sulfate (1.4 g) is added to the aqueous solution in step 2. The mixture is stirred for 20 min at room temperature.
4. 50 mL dichloromethane is added to the solution in step 3. The mixture is stirred for 2 hours at room temperature.
5. The mixture of step 4 is transferred to a separatory funnel and allowed to settle for 3 hours. Two phases are formed, a lower phase of dichloromethane and an upper phase of water.
7. The upper and lower phases in step 5 are separated. The lower dichloromethane phase is recovered and the dichloromethane is evaporated to dryness at room temperature, followed by drying in a vacuum oven for 4 hours at 40° C. A complex of pregabalin-lauryl sulfate (2.1 g) is obtained.

### Example 2

### In Vivo Colonic Absorption Using Flushed Ligated Colonic Model in Rats

An animal model commonly known as the “flush ligated colonic model” or “intracolonic ligated model” was used. Fasted, 0.3-0.5 kg Sprague-Dawley male rats were anesthetized and a segment of proximal colon was isolated. The colon was flushed of fecal materials. The segment was ligated at both ends while a catheter was placed in the lumen and exteriorized above the skin for delivery of test formulation. The colonic contents were flushed out and the colon was returned to the abdomen of the animal. Depending on the experimental set up, the test formulation was added after the segment was filled with 1 mL/kg of 20 mM sodium phosphate buffer, pH 7.4, to more accurately simulate the actual colon environment in a clinical situation.

Rats (n=3) were allowed to equilibrate for approximately 1 hour after surgical preparation and prior to exposure to each test formulation. Gabapentin-lauryl sulfate complex or gabapentin was administered as an intracolonic bolus and delivered at 10 mg gabapentin-lauryl sulfate complex/rat or 10 mg gabapentin/rat. Blood samples obtained from the jugular catheter were taken at 0, 15, 30, 60, 90, 120, 180 and 240 minutes and analyzed for gabapentin concentration. At the end of the 4 hour test period, the rats were euthanized with an overdose of pentobarbital. Colonic segments from each rat were excised and opened longitudinally along the anti-mesenteric border. Each segment was pbserved macroscopically for irritation and any abnormality noted. The excised colons were placed on graph paper and measured to approximate colonic surface area. There was no histopathological change visible to the naked eye in the mucosal of any of the test rats.

A control group of rats (n=3) were treated with gabapentin intravenously, at a dose of 1 mg/rat. Blood samples were withdrawn at the same times indicated above for analysis of gabapentin concentration.

The gabapentin plasma concentration for each test animal, and the average plasma concentration for animals in each test group, are shown in Tables A-C. FIG. 5 shows the average gabapentin concentration in each test group as a function of time.

TABLE A
Gabapentin - Intravenous Administration

| Time | Rat1 | Rat2 | Rat3 | Average | Standard |
|---|---|---|---|---|---|
| (h) | (ng/mL) | (ng/mL) | (ng/mL) | (ng/mL) | Deviation |
| 0 | 0 | 0 | 0 | 0.0 | 0.0 |
| 0.03 | 3340 | 2170 | 2330 | 2613.3 | 634.4 |
| 0.167 | 1420 | 1280 | 1080 | 1260.0 | 170.9 |
| 0.5 | 933 | 868 | 855 | 885.3 | 41.8 |
| 1 | 878 | 867 | 779 | 841.3 | 54.3 |
| 1.5 | 714 | 770 | 648 | 710.7 | 61.1 |
| 2 | 573 | 690 | 518 | 593.7 | 87.8 |
| 3 | 505 | 558 | 415 | 492.7 | 72.3 |

TABLE B
Gabapentin - Colonic Intubation

| Time | Rat1 | Rat2 | Rat3 | Average | Standard |
|---|---|---|---|---|---|
| (h) | (ng/mL) | (ng/mL) | (ng/mL) | (ng/mL) | Deviation |
| 0 | 0 | 0 | 0 | 0.0 | 0.0 |
| 0.25 | 40.6 | 53.8 | 32 | 42.1 | 11.0 |
| 0.5 | 82.5 | 100 | 64.8 | 82.4 | 17.6 |
| 1 | 189 | 210 | 83.8 | 160.9 | 67.6 |
| 1.5 | 266 | 240 | 78.6 | 194.9 | 101.5 |
| 3 | 413 | 265 | 92.9 | 257.0 | 160.2 |
| 4 | 279 | 322 | 94.7 | 231.9 | 120.7 |

TABLE C
Gabapentin Lauryl Sulfate - Colonic Intubation

| Time | Rat1 | Rat2 | Rat3 | Average | Standard |
|---|---|---|---|---|---|
| (h) | (ng/mL) | (ng/mL) | (ng/mL) | (ng/mL) | Deviation |
| 0 | 0 | 0 | 0 | 0.0 | 0.0 |
| 0.25 | 2160 | 2380 | 2790 | 2443.3 | 319.7 |
| 0.5 | 2110 | 2710 | 4440 | 3086.7 | 1209.8 |
| 1 | 2990 | 3280 | 3960 | 3410.0 | 497.9 |
| 1.5 | 3050 | 3270 | 3750 | 3356.7 | 358.0 |
| 3 | 2170 | 2410 | 2140 | 2240.0 | 148.0 |
| 4 | 1380 | 1520 | 1380 | 1426.7 | 80.8 |

### Example 3

### In Vivo Absorption

Twenty-eight rats were randomized into seven test groups (n=4). Gagapentin or gabapentin-lauryl sulfate complex, prepared as described in Example 1A, was intubated via catheter into the beginning of the duodenum of rats at dosages of 5 mg/rat, 10 mg/rat, and 20 mg/rat. The remaining test group was given 1 mg/kg gabapentin intravenously.

Blood samples were taken from each animal over a four hour period and analyzed for gabapentin content. The results are shown in Tables D-H and in FIGS. 6A-6C.

TABLE D
Gabapentin lauryl sulfate, duodenal dose 5 mg/rat

| Time | rat1 | rat2 | rat3 | rat4 | | Std |
|---|---|---|---|---|---|---|
| (h) | (ng/mL) | (ng/mL) | (ng/mL) | (ng/mL) | Average | Dev. |
| 0 | 0 | 0 | 0 | 0 | 0 | 0 |
| 0.25 | 1490 | 1410 | 2130 | 2400 | 1857.5 | 484.4 |
| 0.5 | 2690 | 2080 | 3210 | 3700 | 2920 | 695.5 |
| 1 | 2380 | 2720 | 2750 | 4640 | 3122.5 | 1025.5 |
| 1.5 | 2500 | 2620 | 2470 | 4010 | 2900.0 | 742.8 |
| 2 | 1970 | 2740 | 1520 | 3620 | 2462.5 | 921.5 |
| 3 | 1580 | 1670 | 1230 | 2860 | 1835.0 | 709.2 |
| 4 | 967 | 1120 | 696 | 1710 | 1123.25 | 428.8 |

TABLE E
Gabapentin lauryl sulfate, duodenal dose 10 mg/rat

| Time | rat1 | rat2 | rat3 | rat4 | | Std |
|---|---|---|---|---|---|---|
| (h) | (ng/mL) | (ng/mL) | (ng/mL) | (ng/mL) | Average | Dev. |
| 0 | 0 | 0 | 0 | 0 | 0 | 0 |
| 0.25 | 2260 | 2510 | 2440 | 3080 | 2572.5 | 354.3 |
| 0.5 | 3210 | 4010 | 3220 | 4350 | 3697.5 | 574.2 |
| 1 | 3670 | 3150 | 4010 | 4910 | 3935 | 740.0 |
| 1.5 | 2890 | 4590 | 4240 | 6370 | 4522.5 | 1433.3 |
| 2 | 2310 | 3880 | 4200 | 5190 | 3895 | 1194.8 |
| 3 | 1410 | 3630 | 5210 | 3400 | 3412.5 | 1558.7 |
| 4 | 981 | 2230 | 2430 | 1760 | 1850.2 | 644.0 |

TABLE F
Gabapentin lauryl sulfate, duodenal dose 20 mg/rat

| Time | rat1 | Rat2 | rat3 | rat4 | | Std |
|---|---|---|---|---|---|---|
| (h) | (ng/mL) | (ng/mL) | (ng/mL) | (ng/mL) | Average | Dev. |
| 0 | 0 | 0 | 0 | 0 | 0 | 0 |
| 0.25 | 5570 | 4270 | 5910 | 3420 | 4792.5 | 1156.2 |
| 0.5 | 5320 | 4680 | 6410 | 4820 | 5307.5 | 784.7 |
| 1 | 7370 | 6610 | 7000 | 6550 | 6882.5 | 381.4 |
| 1.5 | 6770 | 6820 | 7830 | 8380 | 7450 | 789.3 |
| 2 | 5670 | 6980 | 8100 | 9410 | 7540 | 1593.842 |
| 3 | 3720 | 5970 | 5880 | 7210 | 5695 | 1449.793 |
| 4 | 2570 | 4980 | 3330 | 4060 | 3735 | 1029.061 |

TABLE G
Gabapentin, duodenal dose 5 mg/rat

| | rat1 | rat2 | rat3 | rat4 | | Std |
|---|---|---|---|---|---|---|
| Time (h) | (ng/mL) | (ng/mL) | (ng/mL) | (ng/mL) | Average | Dev. |
| 0 | 0 | 0 | 5.71 | 0 | 1.4275 | 2.855 |
| 0.25 | 3920 | 2590 | 3110 | 4020 | 3410 | 681.8 |
| 0.5 | 7500 | 4420 | 4400 | 6850 | 5792.5 | 1618.3 |
| 1 | 10800 | 7610 | 6350 | 7870 | 8157.5 | 1882.6 |
| 1.5 | 11400 | 8410 | 7260 | 7740 | 8702.5 | 1859.2 |
| 2 | 9390 | 6800 | 9370 | 6670 | 8057.5 | 1528.0 |
| 3 | 6350 | 5830 | 5640 | 5370 | 5797.5 | 413.9 |
| 4 | 4710 | 3490 | 3900 | 3350 | 3862.5 | 611.3 |

TABLE H
Gabapentin, duodenal dose 10 mg/rat

| | rat1 | rat2 | rat3 | rat4 | | Std |
|---|---|---|---|---|---|---|
| Time (h) | (ng/ml) | (ng/ml) | (ng/ml) | (ng/ml) | Average | Dev. |
| 0 | 0 | 0 | 5.62 | 0 | 1.405 | 2.81 |
| 0.25 | 5690 | 2760 | 5740 | 5110 | 4825 | 1406.1 |
| 0.5 | 7560 | 4480 | 8490 | 9260 | 7447.5 | 2096.9 |
| 1 | 7600 | 7320 | missing | 11400 | 8773.333 | 2279.1 |
| 1.5 | 7150 | 6170 | 10500 | 14900 | 9680 | 3943.0 |
| 2 | 8020 | 11000 | 12500 | 14800 | 11580 | 2841.6 |
| 3 | 6580 | 12900 | 9740 | 14100 | 10830 | 3377.8 |
| 4 | 4610 | 12400 | 6820 | 8660 | 8122.5 | 3297.5 |

TABLE F
Gabapentin, duodenal dose 20 mg/rat

| | rat1 | rat2 | rat3 | rat4 | Average | |
|---|---|---|---|---|---|---|
| Time (h) | (ng/mL) | (ng/mL) | (ng/mL) | (ng/mL) | (ng/mL) | Std Dev. |
| 0 | 0 | 0 | 0 | 0 | 0 | 0 |
| 0.25 | 5560 | 6720 | 7910 | 8050 | 7060 | 1164.5 |
| 0.5 | 7360 | 9850 | 13100 | 11800 | 10527.5 | 2498.6 |
| 1 | 7970 | 13500 | 13700 | 15800 | 12742.5 | 3347.4 |
| 1.5 | 10300 | 13400 | 13500 | 16200 | 13350 | 2411.8 |
| 2 | 9530 | 12500 | 14100 | 17600 | 13432.5 | 3362.2 |
| 3 | 6530 | 9070 | 10200 | 16900 | 10675 | 4424.7 |
| 4 | 4370 | 5900 | 6050 | 13900 | 7555 | 4297.6 |

### Example 4

### In Vivo Colonic Absorption Using Flushed Ligated Colonic Model in Rats

An animal model commonly known as the “intracolonic ligated model” is employed. Fasted, 0.3-0.5 kg Sprague-Dawley male rats are anesthetized and a segment of proximal colon is isolated. The colon is flushed of fecal materials. The segment is ligated at both ends while a catheter is placed in the lumen and exteriorized above the skin for delivery of test formulation. The colonic contents are flushed out and the colon is returned to the abdomen of the animal. Depending on the experimental set up, the test formulation is added after the segment is filled with 1 mL/kg of 20 mM sodium phosphate buffer, pH 7.4, to more accurately simulate the actual colon environment in a clinical situation.

Rats (n=3) are allowed to equilibrate for approximately 1 hour after surgical preparation and prior to exposure to each test formulation. Pregabalin-lauryl sulfate complex or pregabalin are administered as an intracolonic bolus and delivered at 10 mg pregabalin/rat. Blood samples obtained from the jugular catheter are taken at 0, 15, 30, 60, 90, 120, 180 and 240 minutes for analysis of pregabalin concentration. At the end of the 4 hour test period, the rats are euthanized with an overdose of pentobarbital. Colonic segments from each rat are excised and opened longitudinally along the anti-mesenteric border. Each segment is observed macroscopically for irritation and any abnormality noted. The excised colons are placed on graph paper and measured to approximate colonic surface area.

A control group of rats (n=3) is treated with pregabalin intravenously, at a dose of 1 mg/rat. Blood samples are withdrawn at the same times indicated above.

### Example 5

### In Vivo Absorption

Twenty-eight rats are randomized into seven test groups (n=4). Pregabalin or pregabalin-lauryl sulfate complex, prepared as described in Example 1B, in water is intubated via catheter into the beginning of the duodenum of rats at dosages of 5 mg/rat, 10 mg/rat, and 20 mg/rat. The remaining test group is given 1 mg/kg pregabalin intravenously.

Blood samples are taken from each animal over a four hour period and analyzed for pregabalin content. The dose, AUC, and bioavailability are determined using similar calculations as used for gabapentin in Example 3.

### Example 6

### Preparation of Dosage Form Comprising a Drug-Transport Moiety Complex

A. Gabapentin-Transport Moiety Complex

A device as shown in FIG. 7 is prepared as follows. A compartment forming composition comprising, in weight percent, 92.25% gabapentin-transport moiety complex, 5% potassium carboxypolymethylene, 2% polyethylene oxide having a molecular weight of about 5,000,000, and 0.5% silicon dioxide are mixed together. Next, the mixture is passed through a 40 mesh stainless steel screen and then dry blended in a V-blender for 30 minutes to produce a uniform blend. Next, 0.25% magnesium stearate is passed through an 80 mesh stainless steel screen, and the blend given an additional 5 to 8 minutes blend. Then, the homogeneously dry blended powder is placed into a hopper and fed to a compartment forming press, and known amounts of the blend compressed into ⅝ inch oval shapes designed for oral use. The oval shaped precompartments are coated next in an Accela-Cota® wall forming coater with a wall forming composition comprising 91% cellulose acetate having an acetyl content of 39.8% and 9% polyethylene glycol 3350. After coating, the wall coated drug compartments are removed from the coater and transferred to a drying oven for removing the residual organic solvent used during the wall forming procedure. Next, the coated devices are transferred to a 50° C. forced air oven for drying about 12 hours. Then, one or more exit ports are formed in the wall of the device using a laser.

B. Pregabalin-Transport Moiety Complex

A device as shown in FIG. 7 is prepared as follows. A compartment forming composition comprising, in weight percent, 92.25% pregabalin-transport moiety complex, 5% potassium carboxypolymethylene, 2% polyethylene oxide having a molecular weight of about 5,000,000, and 0.5% silicon dioxide are mixed together. Next, the mixture is passed through a 40 mesh stainless steel screen and then dry blended in a V-blender for 30 minutes to produce a uniform blend. Next, 0.25% magnesium stearate is passed through an 80 mesh stainless steel screen, and the blend given an additional 5 to 8 minutes blend. Then, the homogeneously dry blended powder is placed into a hopper and fed to a compartment forming press, and known amounts of the blend compressed into ⅝ inch oval shapes designed for oral use. The oval shaped precompartments are coated next in an Accela-Cota® wall forming coater with a wall forming composition comprising 91% cellulose acetate having an acetyl content of 39.8% and 9% polyethylene glycol 3350. After coating, the wall coated drug compartments are removed from the coater and transferred to a drying oven for removing the residual organic solvent used during the wall forming procedure. Next, the coated devices are transferred to a 50° C. forced air oven for drying about 12 hours. Then, one or more exit ports are formed in the wall of the device using a laser.

### Example 7

### Preparation of Dosage Form Comprising a Gabapentin-Transport Moiety Complex

A dosage form, as illustrated in FIG. 9, comprising a layer of gabapentin and a layer of gabapentin-lauryl sulfate complex is prepared as follows.

10 grams of gabapentin, 1.18 g of polyethylene oxide of 100,000 molecular weight, and 0.53 g of polyvinylpyrrolidone having molecular weight of about 38,000 are dry blended in a conventional blender for 20 minutes to yield a homogenous blend. Next, 4 mL denatured anhydrous alcohol is added slowly, with the mixer continuously blending, to the three component dry blend. The mixing is continued for another 5 to 8 minutes. The blended wet composition is passed through a 16 mesh screen and dried overnight at room temperature. Then, the dry granules are passed through a 16 mesh screen and 0.06 g of magnesium stearate are added and all the ingredients are dry blended for 5 minutes. The fresh granules are ready for formulation as the initial dosage layer in the dosage form.

The layer containing gabapentin-lauryl sulfate complex in the dosage form is prepared as follows. First, 9.30 grams of gabapentin-lauryl sulfate complex, prepared as described in Example 1A, 0.50 g polyethylene oxide of 5,000,000 molecular weight, 0.10 g of polyvinylpyrrolidone having molecular weight of about 38,000 are dry blended in a conventional blender for 20 minutes to yield a homogenous blend. Next, denatured anhydrous ethanol is added slowly to the blend with continuous mixing for 5 minutes. The blended wet composition is passed through a 16 mesh screen and dried overnight at room temperature. Then, the dry granules are passed through a 16 mesh screen and 0.10 g magnesium stearate are added and all the dry ingredients were dry blended for 5 minutes.

A push layer comprised of an osmopolymer hydrogel composition is prepared as follows. First, 58.67 g of pharmaceutically acceptable polyethylene oxide comprising a 7,000,000 molecular weight, 5 g Carbopol® 974P, 30 g sodium chloride and 1 g ferric oxide were separately screened through a 40 mesh screen. The screened ingredients were mixed with 5 g of hydroxypropylmethylcellulose of 9,200 molecular weight to produce a homogenous blend. Next, 50 mL of denatured anhydrous alcohol was added slowly to the blend with continuous mixing for 5 minutes. Then, 0.080 g of butylated hydroxytoluene was added followed by more blending. The freshly prepared granulation was passed through a 20 mesh screen and allowed to dry for 20 hours at room temperature (ambient). The dried ingredients were passed through a 20 mesh screen and 0.25 g of magnesium stearate was added and all the ingredients were blended for 5 minutes.

The tri-layer dosage form is prepared as follows. First, 118 mg of the gabapentin composition is added to a punch and die set and tamped, then 511 mg of the gabapentin-lauryl sulfate composition is added to the die set as the second layer and again tamped. Then, 315 mg of the hydrogel composition is added and the three layers are compressed under a compression force of 1.0 ton (1000 kg) into a 9/32 inch (0.714 cm) diameter punch die set, forming an intimate tri-layered core (tablet).

A semipermeable wall-forming composition is prepared comprising 80.0 wt % cellulose acetate having a 39.8 % acetyl content and 20.0 % polyoxyethylene-polyoxypropylene copolymer having a molecular weight of 7680-9510 by dissolving the ingredients in acetone in a 80:20 wt/wt composition to make a 5.0% solids solution. The wall-forming composition is sprayed onto and around the tri-layerd core to provide a 60 to 80 mg thickness semi-permeable wall.

Next, a 40 mil (1.02 mm) exit orifice is laser drilled in the semipermeable walled tri-layered tablet to provide contact of the gabapentin layer with the exterior of the delivery device. The dosage form is dried to remove any residual solvent and water.

### Example 8

### In Vitro Dissolution of a Dosage Form Containing a Gabapentin-Transport Moiety Complex

The in vitro dissolution rates of dosage forms prepared as described in Examples 4 and 5 are determined by placing a dosage form in metal coil sample holders attached to a USP Type VII bath indexer in a constant temperature water bath at 37° C. Aliquots of the release media are injected into a chromatographic system to quantify the amounts of gabapentin (or pregabalin) released into a medium simulating artificial gastric fluid (AGF) during each testing interval.

### Example 9

### Preparation of Dosage Form Comprising a Gabapentin-Transport Moiety Complex

A dosage form as illustrated in FIGS. 10A-10C is prepared as follows. A unit dose for prolonged release of the gabapentin-lauryl sulfate complex is prepared as follows. 200 grams of of gabapentin in the form of gabapentin-lauryl sulfate complex is passed through a sizing screen having 40 wires per inch. 25 grams of hydroxypropyl methylcellulose having a number average molecular weight of 9,200 grams per mole, and 15 grams of hydroxypropyl methylcellulose having a moledular weight of 242,000 grams per mole are passed through a sizing screen having a mesh size of 40 wires per inch. The celluloses each have an average hydroxyl content of 8 weight percent and an average methoxyl content of 22 weight percent. The sized powders are tumble mixed for 5 minutes. Anhydrous ethanol is added to the mixture with stirring until a damp mass is formed. The damp mass is passed through a sizing screen with 20 wires per inch. The resulting damp granules are air dried overnight, and then passed again through the 20 mesh sieve. 2 grams of the tabletting lubricant, magnesium stearate, are passed through a sizing screen with 80 wires per inch. The sized magnesium stearate is blended into the dried granules to form the final granulation.

733 mg portions of the final granulation are placed in die cavities having inside diameters of 0.281 inch. The portions are compressed with deep concave punches under a pressure head of 1 ton, forming longitudinal capsule-shaped tablets.

The capsules are fed into a Tait Capsealer Machine (Tait Design and Machine Co., Manheim, Pa.) where three bands are printed onto each capsule. The material forming the bands is a mixture of 50 wt % ethylcellulose dispersion (Surelease®, Colorcon, West Point, Pa.) and 50 wt % ethyl acrylate methylmethacrylate (Eudragit® NE 30D, RohmPharma, Weiterstadt, Germany). The bands are applied as an aqueous dispersion and the excess water is driven off in a current of warm air. The diameter of the bands is 2 millimeters.

While there has been described and pointed out features and advantages of the invention, as applied to present embodiments, those skilled in the medical art will appreciate that various modifications, changes, additions, and omissions in the method described in the specification can be made without departing from the spirit of the invention.

1. A substance comprised of gabapentin or pregabalin and a transport moiety, said gabapentin or pregabalin and said transport moiety forming a complex.

2. The substance of claim 1, wherein said transport moiety is an alkyl sulfate salt having between 6-12 carbon atoms.

3. The substance of claim 2, wherein said alkyl sulfate salt is a lauryl sulfate salt.

4. A composition, comprising,
a complex comprised of gabapentin or pregabalin and a transport moiety, and
a pharmaceutically acceptable vehicle,
wherein said composition has an absorption in the lower gastrointestinal tract at least 5-fold higher than gabapentin or pregabalin.

5. The composition of claim 4, wherein said transport moiety is an alkyl sulfate salt having between 6-12 carbon atoms.

6. The composition of claim 5, wherein said alkyl sulfate salt is a lauryl sulfate salt.

7. A dosage form comprising the composition of claim 4.

8. A dosage form comprising the substance of claim 1.

9. The dosage form of claim 8, wherein the dosage form is an osmotic dosage form.

10. The dosage form of claim 9, comprised of (i) a push layer; (ii) drug layer comprising a gabapentin-transport moiety complex or a pregabalin-transport moiety complex; (iii) a semipermeable wall provided around the push layer and the drug layer; and (iv) an exit.

11. The dosage form of claim 9, comprised of (i) a semipermeable wall provided around an osmotic formulation a gabapentin-transport moiety complex or a pregabalin-transport moiety complex, an osmagent, and an osmopolymer; and (ii) an exit.

12. The dosage form of claim 9, wherein the dosage form provides a total daily dose of between 200-3600 mg.

13. An improvement in a dosage form comprising gabapentin or pregabalin, the improvement comprising,
a dosage form comprising a complex of gabapentin or pregabalin and a transport moiety associated by a tight-ion pair bond.

14. The improved dosage form of claim 13, wherein said transport moiety is an alkyl sulfate salt having between 6-12 carbon atoms.

15. The improved dosage form of claim 14, wherein said alkyl sulfate salt is a lauryl sulfate salt.

16. A method for administering gabapentin or pregabalin, comprising:
administering the substance of claim 1 to a patient in need thereof.

17. The method of claim 16, wherein said administering is via oral administration.

18. A method of preparing a complex of gabapentin or pregabalin and a transport moiety, comprising
providing gabapentin or pregabalin;
providing a transport moiety;
combining the gabapentin or pregabalin and the transport moiety in the presence of a solvent having a dielectric constant less than that of water;
whereby said combining results in formation of a complex of gabapentin or pregabalin and the transport moiety.

19. The method of claim 18, wherein said combining includes (i) combining the gabapentin or pregabalin and the transport moiety in an aqueous solvent, (ii) adding said solvent having a dielectric constant less than that of water to the aqueous solvent, and (iii) recovering said complex from said solvent.

20. The method of claim 18, wherein said combining comprises contacting in a solvent having a dielectric constant at least two fold lower than the dielectric constant of water.

21. The method of claim 20, wherein said solvent is selected from the group consisting of methanol, ethanol, acetone, benzene, methylene chloride, and carbon tetrachloride.

22. A method of improving G.I. absorption of gabapentin or pregabalin, comprising
providing a complex comprised of gabapentin or pregabalin and a transport moiety; and
administering the complex to a patient.

23. The method of claim 22, wherein the improved absorption comprises improved lower gastrointestinal absorption.

24. The method of claim 22, wherein the improved absorption comprises improved absorption in the upper gastrointestinal tract.
